(19) United States
(12) Patent Application Publication
(10) Pub. No.: US 2024/0285847 A1
(43) Pub. Date: Aug. 29, 2024

(54) LIQUID INGRESS PROTECTION AND DESIGN OF ELECTRONIC CIRCUITRY FOR NEGATIVE PRESSURE WOUND THERAPY SYSTEMS

(71) Applicant: T.J.Smith and Nephew,Limited, Hull (GB)

(72) Inventors: Allan Kenneth Frazer Grugeon Hunt, Beverley (GB); Daniel Jorna, Hull (GB); Jaime Owst, Hull (GB); Felix Clarence Quintanar, Hull (GB); Neil Andrew Ramsbottom, Bolton (GB); Liam Bradley James Thomson, Hull (GB)

(21) Appl. No.: 18/571,520

(22) PCT Filed: Jun. 22, 2022
(86) PCT No.: PCT/EP2022/066993

§ 371 Date: Dec. 18, 2023

(30) Foreign Application Priority Data
Jun. 25, 2021 (GB) 2109148.3
Jun. 25, 2021 (GB) 2109154.1

(51) Int. Cl. A61M 1/00; H05K 1/18

(52) U.S. Cl. CPC A61M 1/962; H05K 1/181; A61M 2205/15; A61M 2205/18; A61M 2205/3317; A61M 2205/3368; A61M 2205/50; A61M 2205/8206; H05K 2201/10053; H05K 2201/10151

(57) ABSTRACT

The approaches described herein can provide mitigation against the risk of one or more malfunctions of electronics of a negative pressure wound therapy device. The one or more malfunctions can include flow reverse current, flow of excessive current, liquid ingress, or inadvertent activation. The approaches described herein can provide protection against a single fault (or higher protection against more than one fault). Advantageously, mitigation against the risk of burning or otherwise causing discomfort to the patient or the risk of fire can be provided.

### TECHNICAL FIELD

Embodiments described herein relate to apparatuses, systems, and methods the treatment of wounds, for example using dressings in combination with negative pressure wound therapy.

### DESCRIPTION OF THE RELATED ART

The treatment of open or chronic wounds that are too large to spontaneously close or otherwise fail to heal by means of applying negative pressure to the site of the wound is well known in the art. Negative pressure wound therapy (“NPWT”) systems currently known in the art commonly involve placing a cover that is impermeable or semi-permeable to fluids over the wound, using various means to seal the cover to the tissue of the patient surrounding the wound, and connecting a source of negative pressure (such as a vacuum pump) to the cover in a manner so that negative pressure is created and maintained under the cover. It is believed that such negative pressures promote wound healing by facilitating the formation of granulation tissue at the wound site and assisting the body's normal inflammatory process while simultaneously removing excess fluid, which may contain adverse cytokines and/or bacteria. However, further improvements in NPWT are needed to fully realize the benefits of treatment.

### SUMMARY

A negative pressure wound therapy system can include a negative pressure source. The negative pressure source can be configured to provide negative pressure to a wound covered by a wound dressing and to aspirate fluid from the wound. The negative pressure wound therapy system can include a circuit board. The circuit board can be configured to support a plurality of electronic components. The plurality of electronic components may include control circuitry. The control circuitry can be configured to control operation of the negative pressure source. The circuit board can be configured to support a plurality of traces electrically connecting the plurality of electronic components. The plurality of traces may include a first set of traces that can be configured to transmit one or more digital signals and a second set of traces that can be configured to transmit one or more analog signals. The control circuitry can be configured to detect a degradation of an electrical signal on at least one trace of the first or second set of traces. The degradation may be caused by one or more of an ingress of liquid onto the circuit board or condensation. The control circuitry can be configured to, responsive to detection of the degradation of the electrical signal caused by one or more of the ingress of liquid or condensation, temporarily or permanently deactivate provision of negative pressure to the wound.

The negative pressure wound therapy system of any of the preceding paragraphs and/or any of the apparatuses, systems, or devices disclosed herein can include one or more of the following features. The negative pressure wound therapy system can include the wound dressing. At least one of the negative pressure source or the circuit board can be configured to be at least partially supported by the wound dressing. The circuit board can be configured to support the negative pressure source. The ingress of liquid can occur as a result of the negative pressure source aspirating fluid from the wound. The degradation of the electrical signal can be caused by a short circuit as a result of the one or more of the ingress of liquid or condensation. The degradation of the electrical signal can be caused by a short circuit between two traces from the first set of traces or between a trace from the first set of traces and ground or power. The two traces from the first set of traces may include portions not coated with waterproof material. The trace from the first set of traces may include a portion not coated with waterproof material. The short circuit may be formed due to liquid coming into contact with the portions not coated with waterproof material or the portion not coated with waterproof material. A trace from the second set of traces can correspond to a feedback line of the negative pressure source. The control circuitry may be configured to detect the degradation of the electrical signal based at least in part on a short across the feedback line. A trace from the second set of traces may be part of a circuitry configured to detect excessive temperature. The control circuitry may be configured to detect the degradation of the electrical signal by determining that the circuitry configured to detect excessive temperature has made an incorrect detection of excessive temperature. The control circuitry may be configured to determine that the circuitry configured to detect excessive temperature has made the incorrect detection of excessive temperature based on processing temperature detected by an additional temperature sensor.

The negative pressure wound therapy system of any of the preceding paragraphs and/or any of the apparatuses, systems, or devices disclosed herein can include one or more of the following features. The negative pressure wound therapy system can include at least one battery. The control circuitry can be configured to drain the at least one battery responsive to the detection of the degradation of the electrical signal. The plurality of electronic components may include a resistor network and a switch. The control circuitry may be configured to open the switch and drain the at least one battery through the resistor network. The plurality of electronic components may include a conductive plane and the switch. The control circuitry may be configured to open the switch and drain the at least one battery into the conductive plane. Temporarily deactivating provision of negative pressure to the wound may include preventing the negative pressure source from being activated to provide negative pressure to the wound for a first time period. The control circuitry may be configured to activate the negative pressure source to provide negative pressure to the wound responsive to expiration of the first time period. The first time period may correspond to a time period for clearing an error caused by one or more of the ingress of liquid or condensation onto the circuit board. Permanently deactivating provision of negative pressure to the wound may include preventing the negative pressure source from being activated to provide negative pressure to the wound. The control circuitry may be configured to detect the degradation of the electrical signal based on data obtained from one or more of a humidity sensor or an electronic fuse (eFuse). The control circuitry may be configured to temporarily or permanently deactivate provision of negative pressure by one or more of blowing a fuse, opening the fuse, opening a switch, or opening a relay. The control circuitry may be configured to generate an alarm responsive to the detection of the degradation of the electrical signal. The control circuitry may include a programmable controller. The programmable controller may be configured to execute instructions that detect the degradation of the electrical signal and, responsive to detection of the degradation of the electrical signal, temporarily or permanently deactivate provision of negative pressure to the wound.

A negative pressure wound therapy system can include a negative pressure source configured to provide negative pressure to a wound covered by a wound dressing. The system can include a printed circuit board. The system can include electronic circuitry supported by the printed circuit board and configured to control operation of the negative pressure source. The system can include at least one fuse configured to provide overcurrent protection. The at least one fuse can be positioned on the printed circuit board and not be surrounded by conductive material. This can prevent formation of a heat sink for the at least one fuse.

The negative pressure wound therapy system of any of the preceding paragraphs and/or any of the apparatuses, systems, or devices disclosed herein can include one or more of the following features. The conductive material can include conductive metal that forms at least one of a ground plane or a power plane supported by the printed circuit board. The printed circuit board can include a top layer and a bottom layer. The at least one fuse may not be surrounded by conductive material on the top layer and the bottom layer.

The negative pressure wound therapy system of any of the preceding paragraphs and/or any of the apparatuses, systems, or devices disclosed herein can include one or more of the following features. The system can include a power source supported by the printed circuit board and configured to provide power to the negative pressure source and the electronic circuitry. The at least one fuse can be interposed between the power source and one or more components of the electronic circuitry. The power source can include a first battery and a second battery. The at least one fuse can include a first fuse interposed between the first battery and the one or more components of the electronic circuitry and a second fuse interposed between the second battery and the one or more components of the electronic circuitry. At least one terminal of the power source can be separated from a proximal conductive component supported by the printed circuit board by a clearance. The at least one terminal of the power source can be electrically connected to the at least one fuse. The clearance can be at least twice of a thickness of the printed circuit board. The system can include a switch connected to the power source and configured to prevent a flow of reverse current. The switch can include a transistor and a body diode connected across the transistor. The switch can be configured to prevent the flow of reverse current into a positive terminal of the power source. The printed circuit board can be flexible.

A negative pressure wound therapy system can include a power source. The system can include a negative pressure source configured to provide negative pressure to a wound covered by a wound dressing. The system can include electronic circuitry configured to receive power from the power source and control provision of power to the negative pressure source. The electronic circuitry can include a first activation control and a second activation control separate from the first activation control. The electronic circuitry can be configured to operate in an inactive mode in which power is not provided to the negative pressure source and in an active mode in which power is provided to the negative pressure source. The electronic circuitry can be configured to, in response to activation of a first activation control, transition to the active mode in which power is provided to the negative pressure source. The electronic circuitry can be configured to, in response to the second activation control being activated, prevent transition to the active mode irrespective of activation of the first activation control.

The negative pressure wound therapy system of any of the preceding paragraphs and/or any of the apparatuses, systems, or devices disclosed herein can include one or more of the following features. The first activation control can be a first tab that is configured to be activated when removed. The second activation control can be a second tab configured to be activated when removed or a jumper configured to be activated when removed. The second activation control can be an optical sensor configured to be activated by exposure to light. Activation of the second activation control can prevents an unintended transition of the electronic circuitry from the inactive mode to the active mode. The unintended transition of the electronic circuitry from the inactive mode to the active mode can be triggered by exposure to one or more of light or increased temperature.

A negative pressure wound therapy system can include a power source. The system can include a negative pressure source configured to provide negative pressure to a wound covered by a wound dressing. The system can include electronic circuitry configured to receive power from the power source and control provision of power to the negative pressure source. The electronic circuitry can include an activation control. The electronic circuitry can be configured to operate in an inactive mode in which power is not provided to the negative pressure source. The electronic circuitry can be configured to, in response to activation of the activation control, operate in an active mode in which power is provided to the negative pressure source. The system can include a controller configured to operate the negative pressure source and cause the electronic circuitry to transition from the active mode to the inactive mode in response to a determination that a duration of time following activation of the controller has not elapsed.

The negative pressure wound therapy system of any of the preceding paragraphs and/or any of the apparatuses, systems, or devices disclosed herein can include one or more of the following features. The system can include an interface configured to cause the electronic circuitry to transition to the inactive mode responsive to receiving a signal from the controller.

A negative pressure wound therapy system can include a power source configured to provide power at first and second levels. The system can include a negative pressure source configured to provide negative pressure to a wound covered by a wound dressing. The negative pressure source can be configured to be powered by power at the second level. The system can include a controller configured to operate the negative pressure source. The controller can be configured to be powered by power at the first level. The system can include a switch configured to receive power at the second level. The switch can be configured to, responsive to a user input, toggle between providing a signal to the controller, the signal causing activation or deactivation of the negative pressure source.

The negative pressure wound therapy system of any of the preceding paragraphs and/or any of the apparatuses, systems, or devices disclosed herein can include one or more of the following features. The controller can be configured to operate in an active mode and a sleep mode in which the controller consumes less power than in the active mode. The controller can be configured to, in the active mode, responsive to receiving a first type of input from the switch, cause the negative pressure source to be activated or deactivated. The controller can be configured to, in the active mode, responsive to receiving a second type of input from the switch, transition to the sleep mode. The second type of input can be different from the first type of input. The switch can be a button. The first type of input can be a press of the button for a first duration. The second type of input can be a press of the button for a second duration different from the first duration. The switch can be the only user interface component that can be manipulated by the user (such as, receive input from the user).

The negative pressure wound therapy system of any of the preceding paragraphs and/or any of the apparatuses, systems, or devices disclosed herein can include one or more of the following features. The system can include the wound dressing and at least one of the negative pressure source, the electronic circuitry, or the controller can be at least partially supported by the wound dressing.

Disclosed herein are methods of operating a negative pressure wound therapy systems of any of the preceding paragraphs and/or any of the devices, apparatuses, or systems disclosed herein.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIGS. 1A-1C illustrate a wound dressing incorporating a source of negative pressure and/or other electronic components within the wound dressing;

FIGS. 2A-2B illustrate an electronics unit that may be incorporated into a wound dressing;

FIG. 3 is an exploded perspective view of an electronics assembly enclosing an electronics unit within a housing;

FIG. 4A illustrates a bottom perspective view of the electronics assembly of FIG. 3;

FIG. 4B illustrates a top perspective view of the electronics assembly of FIG. 3;

FIG. 5A is an exploded view of a wound dressing incorporating an electronics assembly within the wound dressing layers;

FIG. 5B illustrates a cross sectional layout of the material layers of a wound dressing incorporating an electronics assembly within the dressing;

FIGS. 6A-6B and 7A-7B illustrate components of an electronics assembly;

FIG. 8 illustrates a pump outlet mechanism;

FIGS. 9A-9B illustrate implementations for reverse polarity protection;

FIG. 10A illustrates latching circuitry;

FIGS. 10B-10D illustrate latching circuitry improvements;

FIG. 11 illustrates a circuit with a play/pause switch;

FIGS. 12A-12E illustrate thermal isolation for one or more fuses;

FIG. 13 illustrates clearances for tracks not protected by one or more fuses;

FIGS. 14A-14B illustrate forming electrical connections for one or more fuses;

FIG. 15 illustrates electronics and other components of a TNP system;

FIG. 16 illustrates a block diagram of the electronics of a TNP system;

FIG. 17 illustrates a circuit diagram of a portion of a TNP system; and

FIG. 18 illustrates a block diagram of a portion of a TNP system.

### DETAILED DESCRIPTION

Overview Embodiments disclosed herein relate to apparatuses and methods of treating a wound with reduced pressure, including a source of negative pressure and wound dressing components and apparatuses. These apparatuses and components, including but not limited to wound overlays, backing layers, cover layers, drapes, sealing layers, spacer layers, absorbent layers, transmission layers, wound contact layers, packing materials, fillers and/or fluidic connectors are sometimes collectively referred to herein as dressings.

The systems and methods disclosed herein can be used to detect liquid ingress and/or condensation (collectively sometimes referred to as liquid ingress) into wound dressings that include (such as, support) electronics including one or more electronic components. Condensation can be caused by an enclosure not being sufficiently tight allowing some ingress of fluid, and can be exacerbated by one or more of humidity or temperature (such as, in a negative pressure wound therapy system supported by a dressing). Liquid ingress may occur as a result of aspirating fluid from a wound or patient misuse (such as, exposure of the system to fluid). The systems and methods disclosed herein can include control circuitry that causes provision of therapy via a wound dressing. The control circuitry can disable the provision of therapy upon detection of the liquid ingress and/or condensation within the electronics. The control circuitry can subsequently re-enable the provision of therapy or may permanently disable the provision of therapy. The systems and methods disclosed herein can enable the detection of liquid ingress and/or condensation by detecting a degradation in digital and/or analog communications in the electronics. Further, the systems and methods disclosed herein can enable the detection of liquid ingress and/or condensation based on signals received from an electronic component of the one or more electronic components. Liquid ingress and/or condensation can cause one or more malfunctions within the electronics that may result in injuries or other complications for a patient and/or the system. For example, a degradation in electrical communications can be caused, which may lead to an increase in the temperature. This can cause discomfort or injury to the patient (such as, a burn, a fire, etc.) The electronics may become damaged. The systems and methods disclosed herein can further enable mitigation of the degradation of the electrical communications as caused by the liquid ingress and/or condensation (e.g., by generating an alarm, blowing a fuse, etc.) as a result of detecting liquid ingress and/or condensation. This can reduce the risk of causing discomfort or injury to the patient and improve safety and comfort.

It will be appreciated that throughout this specification reference is made to a wound. It is to be understood that the term wound is to be broadly construed and encompasses open and closed wounds in which skin may be torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

It will be understood that embodiments of the present disclosure are generally applicable to use in NPWT or topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, 1013.25 mbar, etc.). Accordingly, a negative pressure value of −X mmHg reflects absolute pressure that is X mmHg below 760 mmHg or, in other words, an absolute pressure of (760−X) mmHg. In addition, negative pressure that is “less” or “smaller” than X mmHg corresponds to pressure that is closer to atmospheric pressure (such as, −40 mmHg is less than −60 mmHg). Negative pressure that is “more” or “greater” than −X mmHg corresponds to pressure that is further from atmospheric pressure (such as, −80 mmHg is more than −60 mmHg). In some cases, local ambient atmospheric pressure is used as a reference point, and such local atmospheric pressure may not necessarily be, for example, 760 mmHg.

The negative pressure range can be approximately −80 mmHg, or between about −20 mmHg and −200 mmHg. Note that these pressures are relative to normal ambient atmospheric pressure, which can be 760 mmHg. Thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the negative pressure apparatus.

The systems and methods disclosed herein relate to the use of a wound dressing. The wound dressing may include one or more electrical components in order to provide a therapy function to a wound. In order to provide the therapy function, the wound dressing may be connected to control circuitry that causes the wound dressing to perform wound therapy (e.g., TNP therapy, ultrasound therapy, compression therapy, light therapy, etc.). The control circuitry can drive the wound dressing by providing a drive signal to the wound dressing that causes the wound dressing to perform wound therapy. Further, the control circuitry can utilize digital and/or analog communications (e.g., the functional degradation or attenuation of digital communications or analog communications) to detect the presence of liquid particles (e.g., manufacturing board residue, bag residue, blood, human exudate, water, condensation, etc.) and/or solid particles (e.g., solder, defective coating, etc.). The liquid particles and the solid particles may be electrically conductive particles that can act as a path to transmit current from a first location to a second location.

Disclosed systems and methods can detect liquid ingress. The term “liquid ingress” may generally be used to describe the ingress of liquid particles and/or solid particles. For example, liquid ingress from a wound of the patient can be detected. Particular portions of the wound dressing and/or the control circuitry may be more susceptible to liquid ingress. For example, an inlet of a negative pressure source may be a high risk area for liquid ingress. The control circuitry can identify a liquid ingress event in response to detecting a degradation in the digital and/or analog communications. The parameters of the detection of liquid ingress can be managed and/or set by a user. For example, a user may be able to set an amount of liquid that would trigger liquid ingress detection. As another example, the user may be able to set an amount of liquid that would trigger mitigation. Mitigation can include one or more of deactivating therapy, generating alarms and/or alerts for the user, reporting the detection (e.g., via wired and/or wireless communications) to the user or a third party. For example, an alert for a user identifying that a liquid ingress event has occurred can be generated. In response, the dressing can be removed from the patient. The detection of liquid ingress can promote patient comfort and safety (for instance, by mitigating the risk of patient burns, risk of fire, discomfort, or the like).

### Wound Dressing

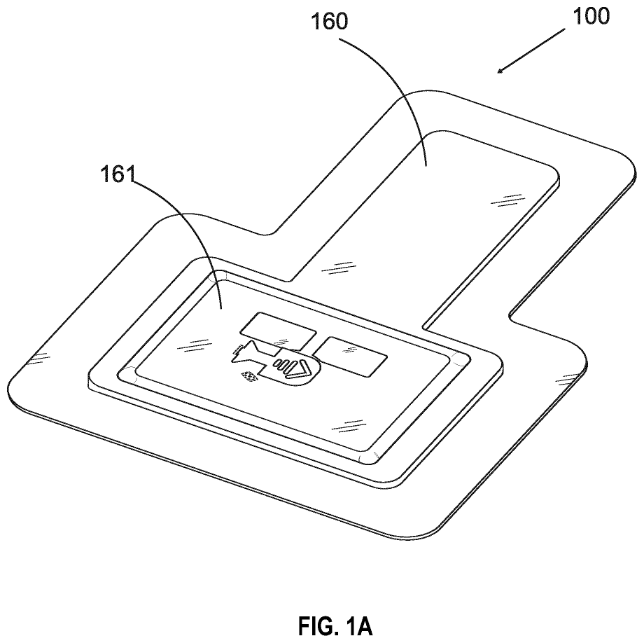

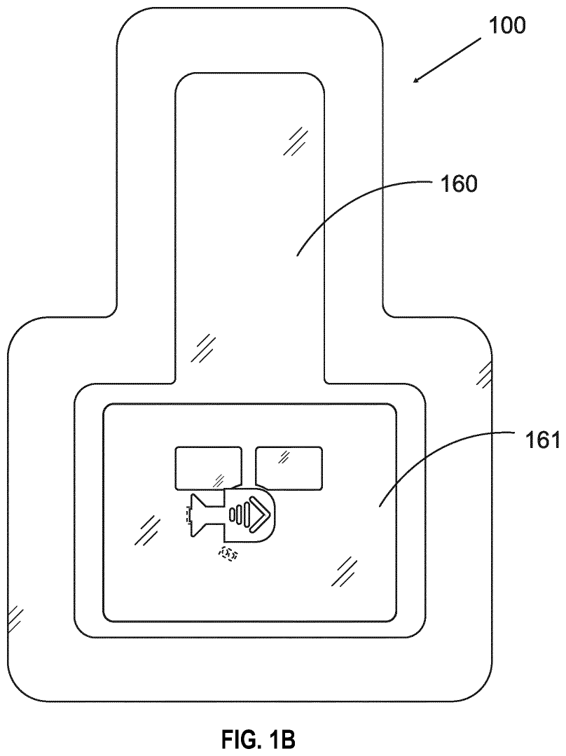

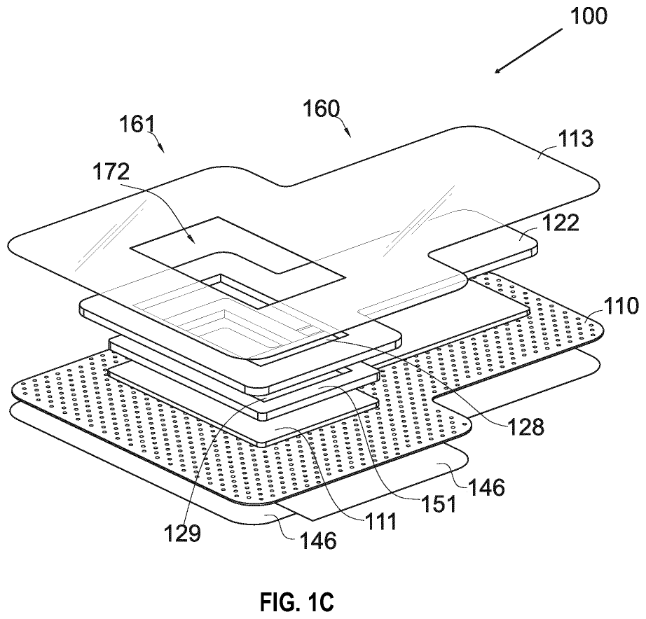

A source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing. The material layers can include a wound contact layer, one or more absorbent layers, one or more transmission or spacer layers, and a backing layer or cover layer covering the one or more absorbent and transmission or spacer layers. The wound dressing can be placed over a wound and sealed to the wound with the pump and/or other electronic components contained under the cover layer within the wound dressing. The dressing can be provided as a single article with all wound dressing elements (including the pump) pre-attached and integrated into a single unit. A periphery of the wound contact layer can be attached to the periphery of the cover layer enclosing all wound dressing elements as illustrated in FIG. 1A-1C.

The pump and/or other electronic components can be configured to be positioned adjacent to or next to the absorbent and/or transmission layers so that the pump and/or other electronic components are still part of a single article to be applied to a patient. The pump and/or other electronics can be positioned away from the wound site. Although certain features disclosed herein may be described as relating to systems and method for controlling operation of a negative pressure wound therapy system in which the pump and/or other electronic components are positioned in or on the wound dressing, the systems and methods disclosed herein are applicable to any negative pressure wound therapy system or any medical device. FIGS. 1A-1C illustrate a wound dressing incorporating the source of negative pressure and/or other electronic components within the wound dressing. FIGS. 1A-1C illustrate a wound dressing 100 with the pump and/or other electronics positioned away from the wound site. The wound dressing can include an electronics area 161 and an absorbent area 160. The dressing can comprise a wound contact layer 110 (not shown in FIGS. 1A-1B) and a moisture vapor permeable film, cover layer or backing layer 113 positioned above the contact layer and other layers of the dressing. The wound dressing layers and components of the electronics area as well as the absorbent area can be covered by one continuous cover layer 113 as shown in FIGS. 1A-1C.

A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. In particular, the transmission layer 111 preferably ensures that an open air channel can be maintained to communicate negative pressure over the wound area even when the absorbent layer has absorbed substantial amounts of exudates. The layer 111 should preferably remain open under the typical pressures that will be applied during negative pressure wound therapy as described above, so that the whole wound site sees an equalized negative pressure. The layer 111 may be formed of a material having a three dimensional structure. For example, a knitted or woven spacer fabric (for example Baltex 7970 weft knitted polyester) or a non-woven fabric could be used.

Further, one or more absorbent layers (such as layers 122, 151) for absorbing and retaining exudate aspirated from the wound can be utilized. A superabsorbent material can be used in the absorbent layers 122, 151. The one or more layers 122, 151 of absorbent material may be provided above the transmission layer 111. Since in use each of the absorbent layers experiences negative pressures, the material of the absorbent layer can be chosen to absorb liquid under such circumstances. The absorbent layers 122. 151 may comprise a composite comprising superabsorbent powder, fibrous material such as cellulose, and bonding fibers. The composite can be an airlaid, thermally-bonded composite.

The electronics area 161 can include a source of negative pressure (such as a pump) and some or all other components of the TNP system, such as power source(s), sensor(s), connector(s), user interface component(s) (such as button(s), switch(es), speaker(s), screen(s), etc.) and the like, that can be integral with the wound dressing. For example, the electronics area 161 can include a button or switch (shown in FIGS. 1A-1B as being covered by a pull tab). The button or switch can be used for operating the pump (such as, turning the pump on/off).

The electronics area 161 of the dressing can comprise one or more layers of transmission or spacer material and/or absorbent material and electronic components can be embedded within the one or more layers of transmission or spacer material and/or absorbent material. The layers of transmission or absorbent material can have recesses or cut outs to embed the electronic components within whilst providing structure to prevent collapse. As shown in FIG. 1C, recesses 128 and 129 can be provided in absorbent layers 151 and 122, respectively.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound. Additionally, the layers can have a proximal wound-facing face referring to a side or face of the layer closest to the skin or wound and a distal face referring to a side or face of the layer furthest from the skin or wound.

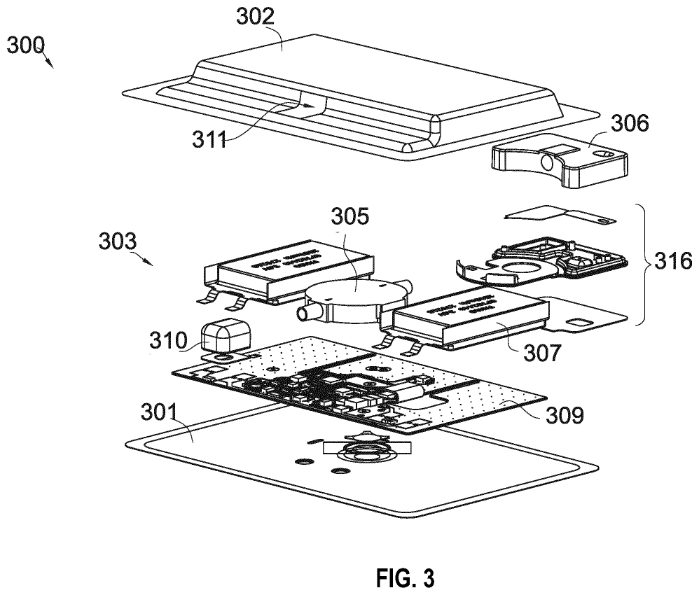

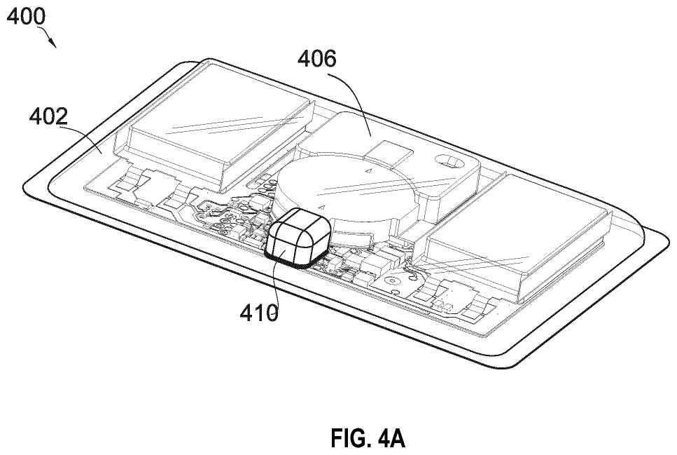

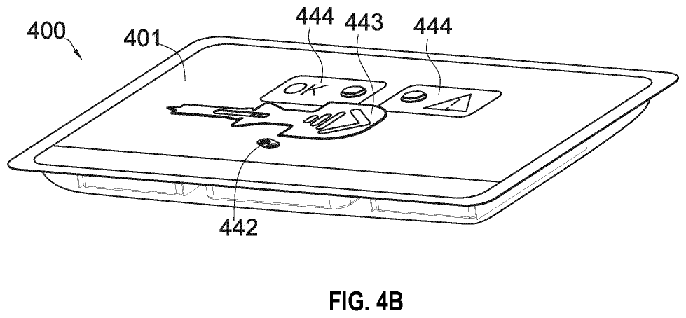

The cover layer may include a cutout 172 positioned over at least a portion of the aperture 128 in the absorbent layer 122 to allow access and fluid communication to at least a portion of the absorbent layers 122 and 151, transmission layer 111, and would contact layer 110 positioned below. An electronics assembly such as described below can be positioned in the apertures 128, 129, and 172 of the first and second absorbent material 151 and 122 and the cover layer 113. The electronics assembly can include a pump, power source, and a printed circuit board as described with reference to FIGS. 3 and 4A-4B.

Before use, the dressing can include one or more delivery layers 146 adhered to the bottom surface of the wound contact layer. The delivery layer 146 can cover adhesive or apertures on the bottom surface of the wound contact layer 110. The delivery layer 146 can provided support for the dressing and can assist in sterile and appropriate placement of the dressing over the wound and skin of the patient. The delivery layer 146 can include handles that can be used by the user to separate the delivery layer 146 from the wound contact layer 110 before applying the dressing to a wound and skin of a patient.

### Electronics Assembly Incorporated Within the Wound Dressing

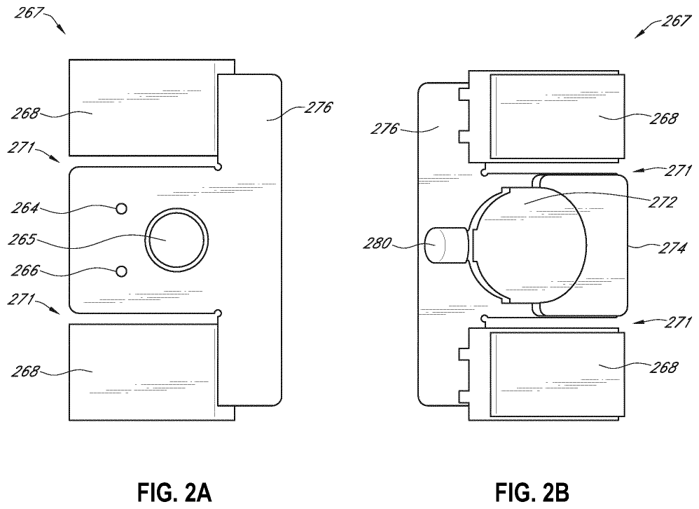

FIGS. 2A-2B illustrate an electronics unit 267 that can be incorporated into a wound dressing. FIG. 2A illustrates the top view of the electronics unit. FIG. 2B illustrates a bottom or wound facing surface of the electronics unit. The electronics unit 267 can include a pump 272 and one or more power sources 268, such as batteries. The electronics unit 267 can include a circuit board 276 configured to be in electrical communication with the pump 272 and/or power source 268. The circuit board 276 can be flexible or substantially flexible.

As illustrated in FIG. 2A, the electronics unit 267 can include single button or switch 265 on the upper surface of the unit. The single button or switch 265 can be used as an on/off button or switch to stop and start operation of the pump and/or electronic components. The electronics unit 267 can also include one or more vents or exhaust apertures 264 on the circuit board 276 for expelling the air exhausted from the pump. As shown in FIG. 2B, a pump outlet exhaust mechanism 274 (sometimes referred to as pump exhaust mechanism or pump outlet mechanism) can be attached to the outlet of the pump 272.

The electronics unit 267 can include a pump inlet protection mechanism 280 as shown in FIG. 2B positioned on the portion of the electronics unit closest to the absorbent area and aligned with the inlet of the pump 272. The pump inlet protection mechanism 280 is positioned between the pump inlet and the absorbent area or absorbent layer of the dressing. The pump inlet protection mechanism 280 can include hydrophobic material to prevent fluid from entering the pump 272. The pump inlet protection mechanism 280 (or any of the inlet protection mechanisms disclosed herein) can include a filter.

The upper surface of the electronics unit 267 can include one or more indicators 266 for indicating a condition of the pump and/or level of pressure within the dressing. The indicators can be small LED lights or other light source that are visible through the dressing components or through holes in the dressing components above the indicators. The indicators can be green, yellow, red, orange, or any other color. For example, there can be two lights, one green light and one orange light. The green light can indicate the device is working properly and the orange light can indicate that there is some issue with the pump (such as, leak, saturation level of the dressing, blockage downstream of the pump, exhaust blockage, low battery, or the like).

The power source 268 can be in electrical communication with the circuit board 276. One or more power source connections are connected to a surface of the circuit board 276. The circuit board 276 can have other electronics incorporated within. For example, the circuit board 276 may support various sensors including, but not limited to, one or more pressure sensors, temperature sensors, optic sensors and/or cameras, and/or saturation indicators.

FIG. 3 illustrates an electronics assembly 300 enclosing an electronics unit within a housing. As illustrated in FIG. 3, the housing of the electronics assembly 300 can include a plate 301 and flexible film 302 enclosing the electronics unit 303 within. The electronics unit 303 can include a pump 305, inlet protection mechanism 310, pump exhaust mechanism 306, power source 307, and circuit board 309. The circuit board 309 can be flexible or substantially flexible.

As is illustrated, the pump exhaust mechanism 306 can be an enclosure, such as a chamber. The electronics unit 303 and pump 305 can be used without the inlet protection mechanism 310. However, the pump exhaust mechanism 306 and the pump 305 can sit within an extended casing 316.

The flexible film 302 can be attached to the plate 301 to form a fluid tight seal and enclosure around the electronic components. The flexible film 302 can be attached to the plate at a perimeter of the plate by heat welding, adhesive bonding, ultrasonic welding, RF welding, or any other attachment or bonding technique.

The flexible film 302 can include an aperture 311. The aperture 311 can allow the inlet protection mechanism 310 to be in fluid communication with the absorbent and/or transmission layers of the wound dressing. The perimeter of the aperture 311 of the flexible film 303 can be sealed or attached to the inlet protection mechanism 310 to form a fluid tight seal and enclosure around the inlet protection mechanism 310 allowing the electronic components 303 to remain protected from fluid within the dressing. The flexible film 302 can be attached to the inlet protection mechanism 310 at a perimeter of the inlet protection mechanism 310 by heat welding, adhesive bonding, ultrasonic welding, RF welding, or any other attachment or bonding technique. The inlet protection mechanism 310 can prevent wound exudate or liquids from the wound and collected in the absorbent area 160 of the wound dressing from entering the pump and/or electronic components of the electronics assembly 300.

The electronics assembly 300 illustrated in FIG. 3 can be incorporated within the wound dressing such that, once the dressing is applied to the body of the patient, air from within the dressing can pass through the inlet protection mechanism 310 to be pumped out toward the pump exhaust mechanism 306 in communication with an aperture in the casing 316 and the circuit board 309 as described herein.

FIGS. 4A-B illustrate an electronics assembly 400 including a pump inlet protection mechanism 410 sealed to the exterior of the flexible film 402, similar to the description with reference to FIG. 3. Also shown is an exhaust mechanism 406, which can be similar to the exhaust mechanism 306.

FIG. 4A illustrates lower, wound facing surface of the electronics assembly 400. FIG. 4B shows an upper surface of the plate 401 (which can face the patient or user) of the electronics assembly 400. The upper surface of the plate 401 can include an on/off (or play/pause) switch or button cover 443 (illustrated as a pull tab), indicators 444, and/or one or more vent holes 442. Removal of the pull tab 443 can cause activation of the electronics assembly 400, such as provision of power from the power source to the electronics assembly. Further details of operation of the pull tab 443 are described in PCT International Application No. PCT/EP2018/079745, filed Oct. 30, 2018, titled “SAFE OPERATION OF INTEGRATED NEGATIVE PRESSURE WOUND TREATMENT APPARATUSES,” which is incorporated by reference in its entirety herein.

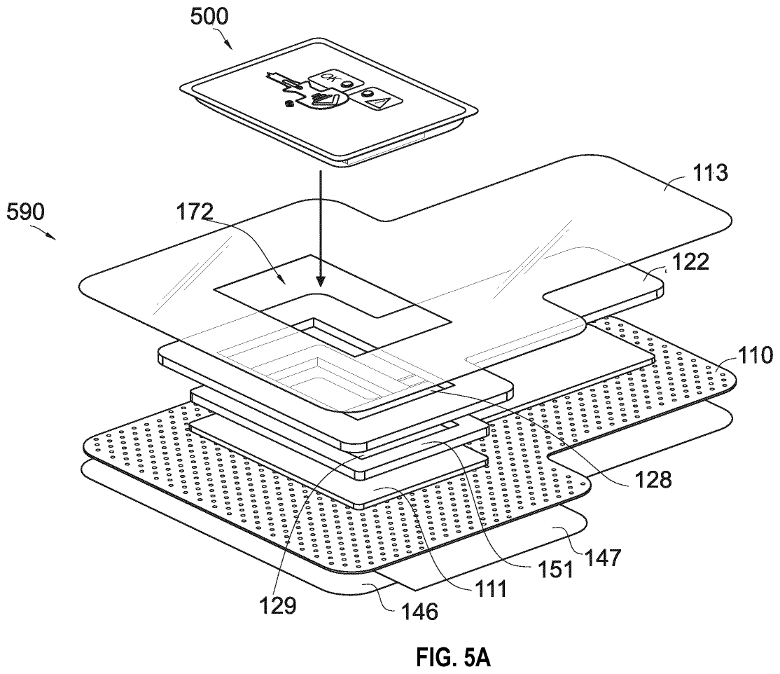

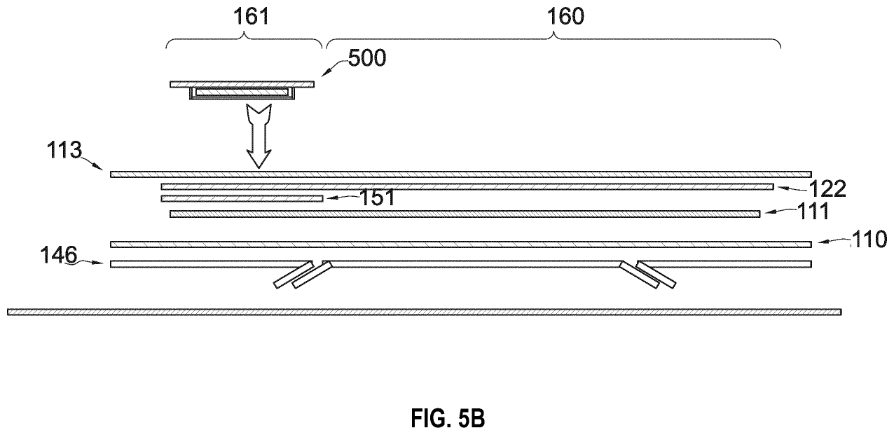

The electronics assembly 400 with the pump inlet protection mechanism 410 extending from and sealed to the film 402 can be positioned within the aperture 172 in the cover layer 113 and absorbent layer(s) (122, 151) as shown in FIG. 1C. The perimeter of the electronics assembly 400 can be sealed to a top surface of the outer perimeter of the aperture 172 in the cover layer 113 as shown in FIGS. 1C and described in more detail with reference to FIG. 5A-5B herein. The electronics assembly 400 can be sealed to the cover layer 113 with a sealant gasket, adhesive, heat welding, adhesive bonding, ultrasonic welding, RF welding, or any other attachment or bonding technique. The electronics assembly 400 can be permanently sealed to the cover layer 113 and could not be removed from the cover layer without destroying the dressing.

The electronics assembly 400 can be utilized in a single dressing and disposed of with the dressing. In some cases, the electronics assembly 400 can be utilized in a series of dressings.

FIG. 5A illustrates a wound dressing, such as the one in FIG. 1C, incorporating an electronics assembly 500 within the wound dressing layers 590. FIG. 5B illustrates a cross-sectional view of the wound dressing incorporating the electronics assembly of FIG. 5A. The electronics assembly 500 can be provided within the aperture 172 in the cover layer and apertures 129 and 128 in the first and second absorbent layers 122, 151. The electronics assembly 500 can seal to the outer perimeter of the aperture 172 of the cover layer. The dressing can comprise a wound contact layer 110 and a moisture vapor permeable film, cover layer or backing layer 113 positioned above the contact layer 110 and other layers of the dressing. A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. Further, one or more absorbent layers (such as layers 122, 151) for absorbing and retaining exudate aspirated from the wound can be utilized. The one or more layers 122, 151 of absorbent material may be provided above the transmission layer 111. There may be a small aperatured absorbent layer 151 and a large aperture absorbent layer 122. The small apertured absorbent layer 151 can be positioned on top of the large apertured absorbent layer 122. In some cases, the small apertured absorbent layer 151 can be positioned below of the large apertured absorbent layer 122. Before use, the dressing can include one or more delivery layers 146 adhered to the bottom surface of the wound contact layer. The delivery layer 146 can cover adhesive or apertures on the bottom surface of the wound contact layer 110.

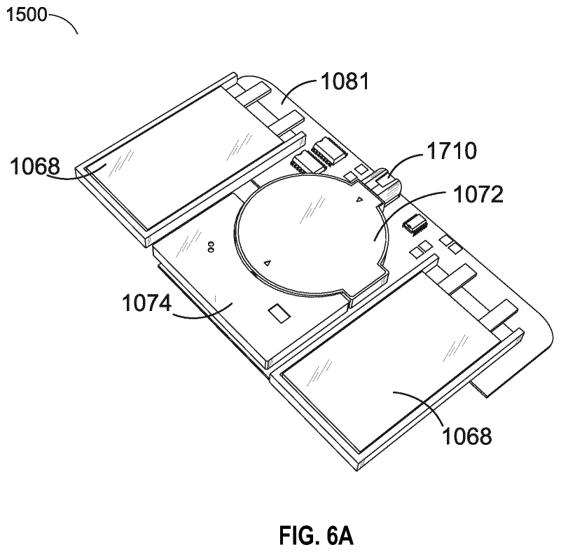

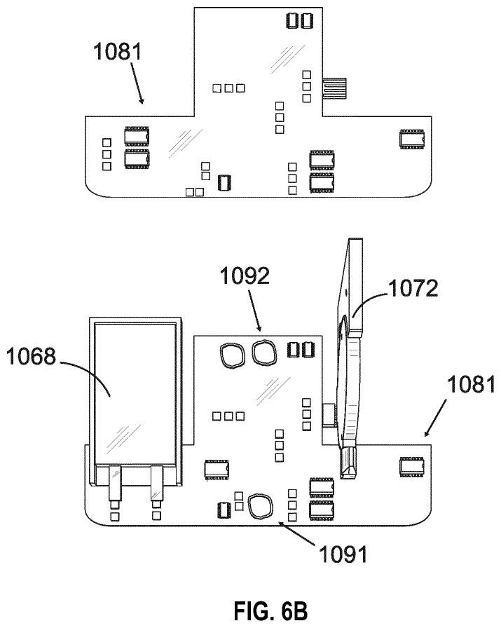

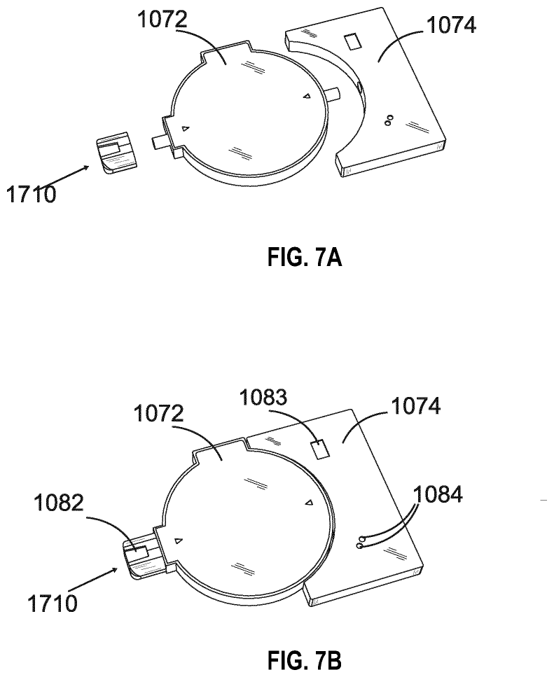

FIGS. 6A-6B and 7A-7B illustrate an electronics assembly 1500 with a pump inlet protection mechanism 1710 and pump exhaust mechanism 1074 on a pump 1072. The assembly 1500 can include cavities 1082 and 1083 (shown in FIGS. 7A-7B) on the pump inlet protection mechanism 1710 and pump exhaust mechanism 1074, respectively. The inlet protection and pump exhaust mechanisms can be adhered to the inlet and the outlet of the pump as described herein. The assembly 1500 can be assembled using an adhesive and allowed to cure prior to incorporating into the electronics assembly.

The pump inlet can be covered or fitted with a pump inlet protection mechanism 1710. The pump inlet protection 1710 can be pushed onto the pump inlet as illustrated by the arrows in FIG. 7A. This can be a friction fit. The port of the pump inlet protection 1710 that receives a portion of the pump inlet can be sized and shaped to be a complementary fit around the pump inlet. The pump inlet protection 1710 can be bonded onto the pump inlet using a silicone sealant or any other sealant or sealing technique. FIG. 7B illustrates the pump inlet protection mechanism 1710 covering the pump inlet and the pump exhaust mechanism 1074 covering the pump outlet. The pump exhaust mechanism 1074 can include one or more apertures or vents 1084 to allow gas aspirated by the pump to be exhausted from the pump exhaust mechanism 1074. In some cases, a non-return valve and/or filter membrane of the pump exhaust mechanism is included in the pump exhaust mechanism 1074.

FIGS. 7A-7B illustrate the pump inlet protection mechanism 1710 and pump exhaust mechanism 1074 with cavities 1082 and 1083. A pump assembly including the pump inlet protection mechanism 1710 and pump exhaust mechanism 1074 can be placed over the surface of a circuit board 1081. When the pump assembly is in contact with the surface of the circuit board 1081, the cavities 1082 and 1083 can at least partially enclose sensors on the circuit board 1081, for example, pressure sensors 1091 and 1092 on the circuit board 1081, as illustrated in FIG. 6B.

The pressure sensors 1091 and 1092 illustrated in FIG. 6B can be used to measure and/or monitor the pressure level at the wound and atmospheric pressure. The pressure sensor 1091 can be used to measure and/or monitor pressure at the wound (such as, underneath the wound dressing), which can be accomplished by measuring and/or monitoring pressure in a fluid flow path connecting the negative pressure source or pump 1072 and the wound. The pressure sensor 1091 can measure and/or monitor pressure in the cavity 1082 of the pump inlet protection mechanism 1710 shown in FIGS. 7A-7B. A power source 1068 (illustrated as two batteries in FIG. 6A) can provide power to the negative pressure source 1072 and the electronics.

The pressure sensor 1092 can be used to measure and/or monitor pressure external to the wound dressing. The pressure sensor 1092 can measure and/or monitor pressure in the cavity 1083 of the pump exhaust mechanism 1074 shown in FIGS. 7A-7B. The pressure sensor 1092 can measure pressure external to the wound dressing, which can be relative atmospheric pressure since the atmospheric pressure varies depending on, for instance, an altitude of use or pressurized environment in which the TNP apparatus may be used. These measurements can be used to establish a desired negative pressure differential (or set point) at the wound relative to the external pressure.

The circuit board 1081 (including any of the circuit boards described herein) can include control circuitry, such as one or more processors or controllers, that can control the supply of negative pressure by the negative pressure source 1072 according at least to a comparison between the pressure monitored by the pressure sensor 1091 and the pressure monitored by the pressure sensor 1092. Control circuitry can operate the negative pressure source 1072 in a first mode (that can be referred to as an initial pump down mode) in which the negative pressure source 1072 is activated to establish the negative pressure set point at the wound. The set point can be set to, for example, a value in the range between about −70 mmHg to about −90 mmHg, among others. Once the set point has been established, which can be verified based on a difference between pressure measured by the pressure sensor 1091 (or wound pressure) and pressure measured by the pressure sensor 1092 (or external pressure), control circuitry can deactivate (or pause) operation of the negative pressure source 1072. Control circuitry can operate the negative pressure source 1072 is a second mode (that can be referred to as maintenance pump down mode) in which the negative pressure source 1072 is periodically activated to re-establish the negative pressure set point when the wound is depressurized as a result of one or more leaks. Control circuitry can activate the negative pressure source 1072 in response to the pressure at the wound (as monitored by the pressure sensor 1091) becomes more positive than a negative pressure threshold, which can be set to the same negative pressure as the set point or lower negative pressure.

Embodiments of the wound dressings, wound treatment apparatuses and methods described herein may also be used in combination or in addition to one or more features described in PCT International Application No. PCT/EP2017/060464, filed May 3, 2017, titled NEGATIVE PRESSURE WOUND THERAPY DEVICE ACTIVATION AND CONTROL, U.S. Pat. Nos. 8,734,425, and 8,905,985, each of which is hereby incorporated by reference in its entirety herein.

One or more self-adhesive gaskets can be applied to the pump inlet protection mechanism 1710 and pump exhaust mechanism 1074 to seal the cavities 1082 and 1083 of the pump inlet and pump exhaust around sensors on the circuit board 1081 and to seal around the exhaust mechanism vent(s) and corresponding vent(s) in the circuit board 1081 (as described herein). A pre-formed adhesive sheet can be used to form the sealing gaskets between the cavities 1082 and 1083 of the pump inlet and pump exhaust mechanisms and sensors on the circuit board 1081 and between the exhaust mechanism vent(s) and vent(s) in the circuit board 1081. In some cases, an adhesive can be used to seal the cavities 1082 and 1083 of the pump inlet protection 1710 and pump exhaust mechanism 1074 around sensors on the circuit board 1081 and to seal around the exhaust mechanism vent(s) 1084 and corresponding vent(s) in the circuit board. As described herein, the electronics assembly 1500 can be embedded within layers of the dressing, such as in cutouts or recesses into which the electronics assembly can be placed.

The pump inlet protection mechanism 1710 can provide a large surface area available for vacuum to be drawn by the inlet of the pump. A pump inlet (shown as rounded protrusion in FIG. 7A) can fit within a recess in the pump inlet protection mechanism 1710. The pump inlet can be attached by friction fit and/or form a complementary fit to the recess of the pump inlet protection mechanism.

The pump inlet protection mechanism 1710 can allow air or gas to pass through, but can block liquid from reaching the negative pressure source. The pump inlet protection mechanism 1710 can include a porous material. The pump inlet protection mechanism 1710 can comprise one or more porous polymer molded components. The pump inlet protection mechanism 1710 can include hydrophobic or substantially hydrophobic material. Material included in the pump inlet protection mechanism 1710 can have a pore size in the range of approximately 5 microns to approximately 40 microns. In some cases, the pore size can be approximately 10 microns. The pump inlet protection mechanism 1710 can include a polymer that can be one of hydrophobic polyethylene or hydrophobic polypropylene. In some cases, the pump inlet protection mechanism can include a Porvair Vyon material with a pore size of 10 microns. Any of the pump inlet protection mechanism described herein can include one or more features of the pump inlet protection mechanism 1710.

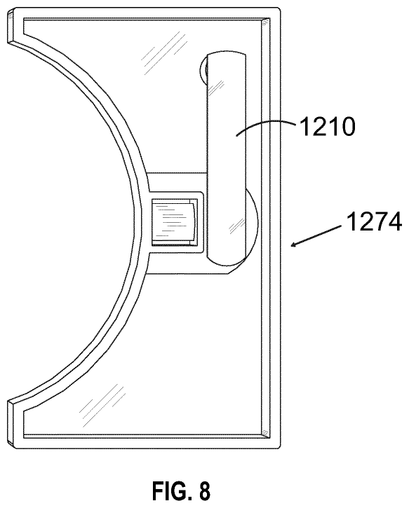

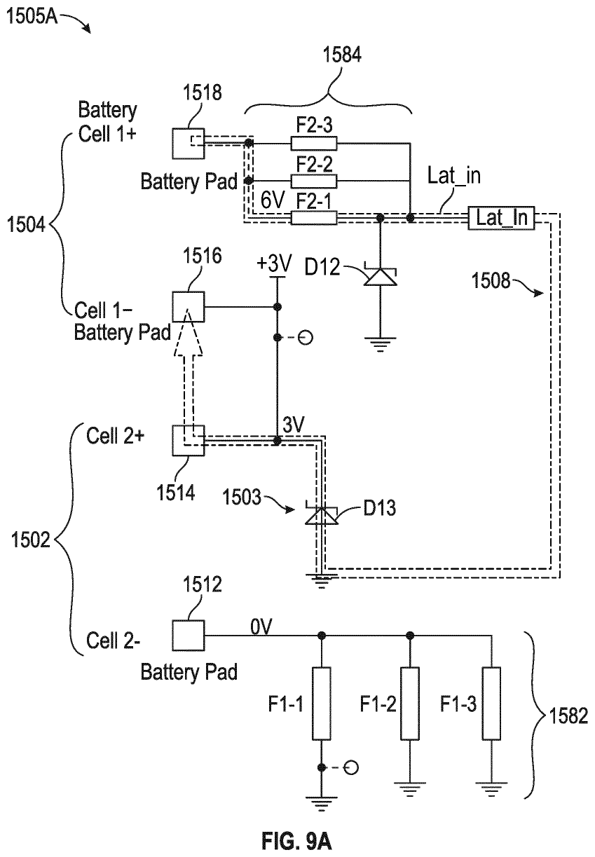

The pump exhaust mechanism 1074 (or any of the pump exhaust or outlet mechanisms described herein) can include a check valve or a non-return valve 1210 as shown in FIG. 8. The non-return valve 1210 can be any suitable mechanical one-way valve, such as, for example, a reed valve, a duckbill valve, a ball valve, a loose leaf valve or an umbrella valve, among others. The non-return valve can be similar to any of the non-return valves described in PCT International Application No. PCT/EP2017/055225, filed Mar. 6, 2017, titled WOUND TREATMENT APPARATUSES AND METHODS WITH NEGATIVE PRESSURE SOURCE INTEGRATED INTO WOUND DRESSING, which is incorporated by reference herein in its entirety. The pump exhaust mechanism 1074 can be bonded to the outlet of the pump using a sealant, for example a silicone sealant. The outlet or exhaust of the pump exhaust mechanism 1074 can include an antimicrobial film and/or other filter membrane that filters gas exhausted outside the NPWT system, such as to the atmosphere. As illustrated, pump exhaust mechanism 1074 can be an enclosure or chamber that is substantially sealed to prevent ingress of gas or fluid other than through the vent(s) 1084.

Any of the embodiments described herein can additionally or alternatively include one or more features described in International Application No. PCT/EP2018/074694, filed Sep. 13, 2018, titled NEGATIVE PRESSURE WOUND TREATMENT APPARATUSES AND METHODS WITH INTEGRATED ELECTRONICS, International Application No. PCT/EP2018/074701, filed Sep. 13, 2018, titled NEGATIVE PRESSURE WOUND TREATMENT APPARATUSES AND METHODS WITH INTEGRATED ELECTRONICS, International Application No. PCT/EP2018/079345, filed Oct. 25, 2018, titled NEGATIVE PRESSURE WOUND TREATMENT APPARATUSES AND METHODS WITH INTEGRATED ELECTRONICS, International Application No. PCT/EP2020/056317, filed Mar. 10, 2020, titled EXHAUST BLOCKAGE DETECTION FOR NEGATIVE PRESSURE WOUND TREATMENT APPARATUSES, each of which is incorporated by reference herein in its entirety.

### Reverse Polarity Protection

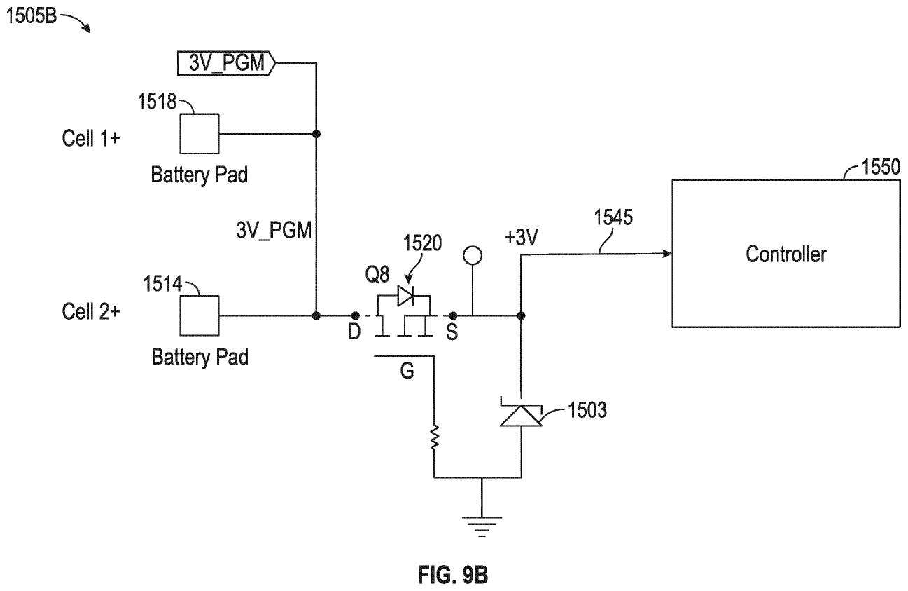

It can be important to protect any of the electronics described herein, such as the electronics unit 267 or the electronics assembly 1500, from reverse current. Reverse current can damage one or more electronic components, which can cause one or more of unsafe provision of therapy or interruption of therapy. As described herein, reverse current can be caused by flexing of a circuit board of the electronics unit or electronics assembly. FIG. 9A illustrates a circuit 1505A in which reverse current 1508 may flow. The circuit 1505A can include two battery cells 1502 and 1504 (which can correspond to the power source 1068). In some cases, the battery cells 1502 and 1504 can be connected in series to increase output voltage. For example, each of the battery cells 1502 and 1504 can be a 3V cell, and the combined output of both cells can be 6V. The combined output of both cells can be sometimes referred to as high voltage. Output of one of the battery cells, such as the battery cell 1502, can be referred to as low voltage (such as, 3V). In some cases, output of one of the battery cells (or low voltage) can provide power to one or more electronic components, such to a controller via the connection 1545 as illustrated in FIG. 9B. Low voltage power connection 1545 can be at the same electrical potential as the LAT_IN terminal described herein. Output of both of the battery cells (or high voltage), which may be enabled by a latching circuit as described herein, can power one or more other electronic components, such as the negative pressure source. Reverse current 1508 may undesirably flow from a positive terminal of one of the battery cells into another terminal of one of the battery cells (which can be a positive terminal, as illustrated in FIG. 9A).

Battery cell 1502 can have terminals 1514 and 1512 (which may be positive and negative terminals, respectively). Battery cell 1504 can have terminals 1518 and 1516 (which may be positive and negative terminals, respectively). A fuse 1582 can protect the circuit 1505A from excessive current being provided by one or more of the battery cells (such as, the battery cell 1502 or both battery cells 1502 and 1504). While multiple fuses 1582 are illustrated in FIG. 9A, only one fuse would be populated in the circuit 1505A during manufacturing. The multiple fuses (such as, three fuses) illustrate three possible fuse footprints formed on a circuit board from which a single suitable fuse 1582 can be selected during manufacturing. A fuse 1584 can protect the circuit 1505A from excessive current being provided by both battery cells. As described herein, even though multiple fuses 1584 are illustrated in FIG. 9A, only one fuse 1584 would be populated. The fuses 1582 and 1584 can be connected to the terminals of the respective battery cells 1502 and 1584 prior to any other electronic components being connected to the battery cells.

The components of the circuit 1505A can be supported by the circuit board. As described herein (such as, with respect to the circuit board 276), the circuit board can be flexible or substantially flexible to accommodate positioning on the patient and wearing by the patient. Flexing of the board can result in shorting the circuit ground connection to the high voltage supply rail (connected to the positive terminal 1518), which can cause the fuse 1582 to open (or blow). As illustrated, a reverse current path can be created in which the current flows into the positive terminal of any of the battery cells 1504 or 1502. Reverse current 1508 can flow from the positive terminal of the high voltage supply rail (such as, the positive terminal 1518 of the battery cell 1504), through a diode 1503, into the positive terminal of the low voltage supply rail (such as, the positive terminal 1514 of the battery cell 1502). The diode 1503 can be configured to provide protection against electrostatic discharge. The diode 1503 can be a Zener diode. In some cases, the fuse 1582 may not protect against reverse current 1508 even after it has been blown. The flow of reverse current may be undesirable, for example, because it can increase temperature or damage of one or both of the battery cells or any of the other circuit components (that can be supported by a wound dressing positioned on the patient), which can cause discomfort to the patient, burn the patient, or otherwise compromise patient comfort or safety as well as start a fire.

With reference to FIG. 9B, a switch 1520 can provide reverse current (or reverse polarity) protection in the illustrated circuit 1505B. As shown, the switch 1520 can be a transistor (such as, p-channel FET or a PNP transistor) with a body diode connected in parallel across the transistor. Under a normal operating condition, the body diode would be forward-biased (or conducting), which would place the source terminal (S) of the switch 1520 at about 2.4V and the gate terminal (G) at about 0V. As a result, the switch 1520 would be turned on, thereby allowing the current to flow across the transistor (whose internal resistance can be relatively small, such as about 50 mOhm or less or more) and bypass the body diode. Under a fault condition (such as, when the circuit ground connection is shorted to the high voltage supply rail, as described herein), the gate terminal (G) voltage would be greater than or equal to the source terminal (S) voltage. As a result, the switch 1520 would be turned off, which can block the flow or the reverse current. In some cases, the body diode can block the flow of the reverse current (for example, since the body diode would be reverse-biased). Accordingly, the switch 1520 and the body diode can provide reverse polarity protection without incurring a diode forward voltage drop. Thereby, full power can be supplied to the one or more electronic components, such as the controller 1550.

In some instances, a diode (such as, a Schottky diode) or a transistor with a resistor can be used as the switch to provide reverse polarity protection (for instance, instead of the transistor and body diode illustrated in FIG. 9B). A Schottky diode may have a low forward voltage drop. As result, when such Schottky diode is forward-biased, less energy would be wasted as heat and efficient supply of power to the one or more electronic components can be achieved.

### Improved Latching Circuitry

As described herein, a play/pause switch (such as, the switch 265) can be configured to start and pause provision of negative pressure wound therapy. It may be advantageous to prevent inadvertent activation of the electronics (which can lead to inadvertent activation of therapy) during manufacturing, transportation, or storage for various reasons, including preserving the capacity of power source, preventing initiation of an end of life countdown for devices that are configured to provide therapy for a limited duration of time (such as, 7 days), or preventing an accident during manufacturing (such as, generating a spark that may inflame gas used for sterilization). To prevent inadvertent activation, the electronics can include circuitry for isolating at least some electronic components from a power source. For instance, such circuitry can isolate the negative pressure source from a high voltage power source (such as, 6V source). As described herein, an activation switch (such as, the pull tab 443) can be positioned on the exterior surface for easy access by the user. As described herein, removal of the pull tab can cause activation of the electronics.

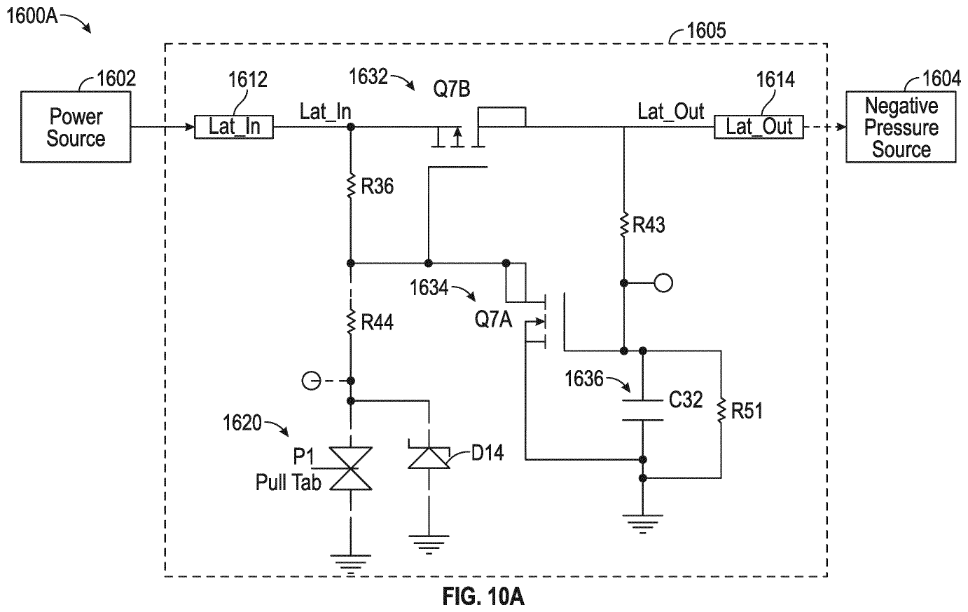

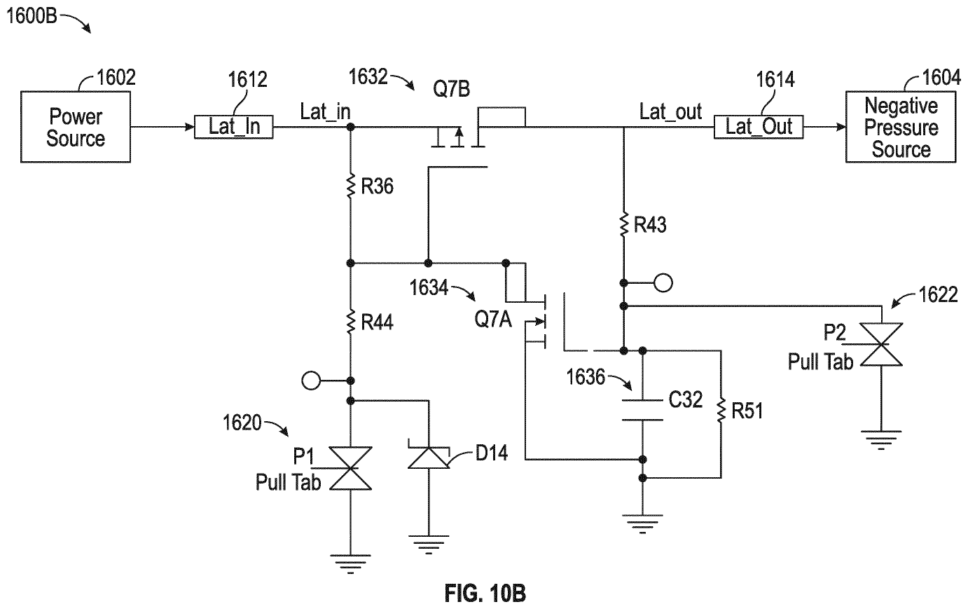

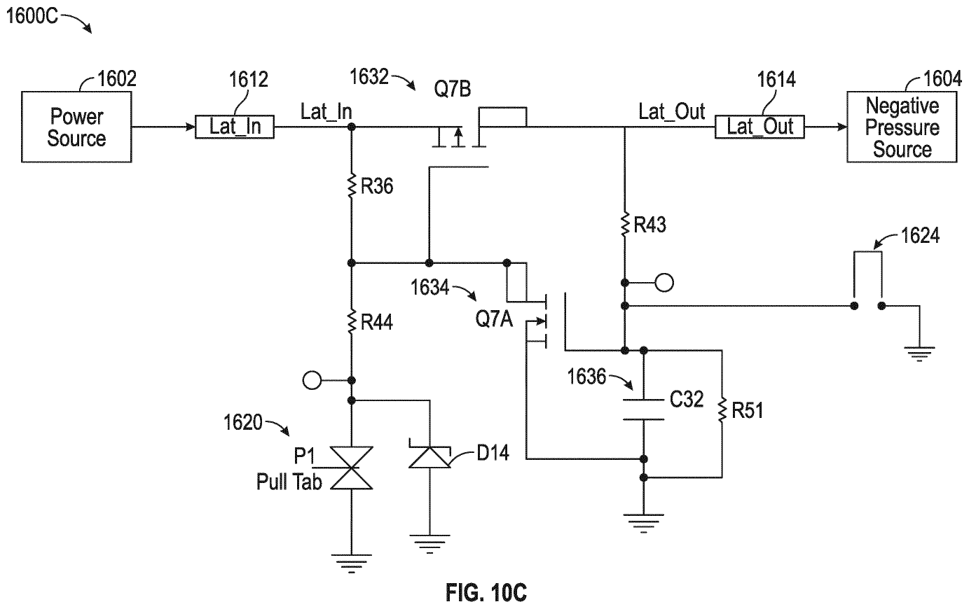

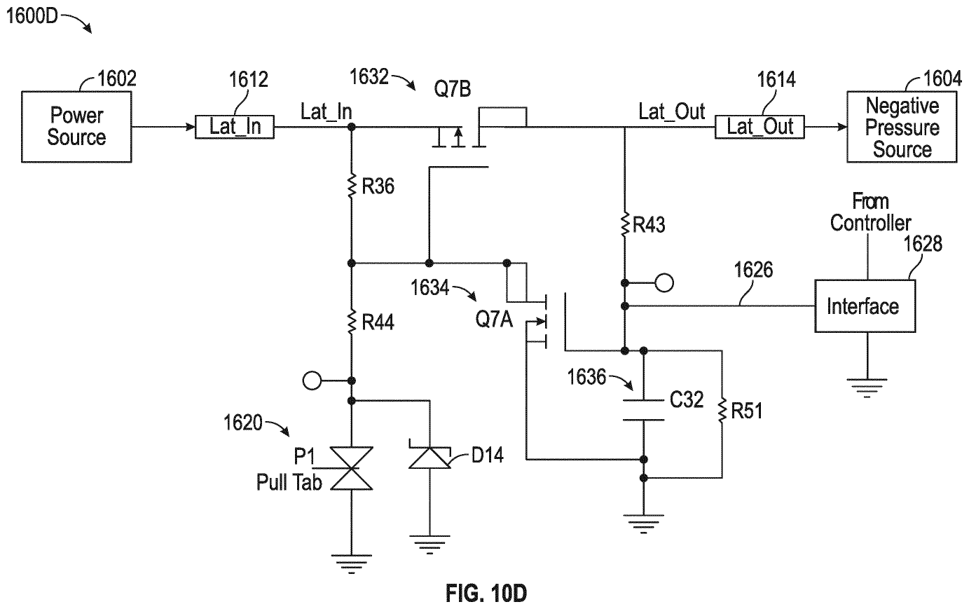

FIG. 10A illustrates circuitry 1600A that can isolate the negative pressure source. The circuitry 1600A can include a power source 1602 and pins or terminals LAT_IN 1612 (representing “latching circuit pin in”) and LAT_OUT 1614 (representing “latching circuit pin out”). The power source 1602 can be connected terminal 1612. The power source 1602 can correspond to the output of the battery cells 1502 and 1504 connected in series and provide high voltage. Latching circuitry 1605 can be interposed between the power source 1602 and one or more electronic components that draw power (such as, current) from the power source 1602. As illustrated, a negative pressure source 1604 can draw power from the power source 1602 and be connected to the LAT_OUT terminal 1614. When the latching circuitry 1605 is in the “on” state (or activated), current can flow between the terminals 1612 and 1614. When the latching circuitry 1605 is in the “off” state (or deactivated), current would not flow between the terminals 1612 and 1614 regardless of the state of the play/pause switch.

The latching circuitry 1605 can include an activation switch or control 1620 (such as, a pull tab). When the activation control 1620 has not been activated (such as, the pull tab has not removed), switches 1632 and 1634 (shown as transistors) are off such that no current flows between the terminals 1612 and 1614. When the activation control 1620 has been activated (for example, the pull tab has been removed), current from the power source 1602 can flow (for example, to the ground). This can cause the switch 1632 (such as, a p-channel FET or a PNP transistor) to turn on, which allows current to flow between the terminals 1612 and 1614. Turning on the switch 1632 can cause the switch 1634 (such as, n-channel FET or an NPN transistor) to turn on. The gate of the switch 1636 can be connected to a capacitor 1636, which may become charged as a result of the flow of current between the terminals 1612 and 1614. As long as the capacitor 1636 remains charged, the switch 1636 can remain turned on, which in turn can maintain the switch 1632 being turned on. As a result, the latching circuitry 1605 can be a self-latching circuitry. The latching circuitry 1605 can operate such that once the activation control 1620 has been activated, the circuitry 1605 remains activated allowing current to flow between the terminals 1612 and 1614.

Once the latching circuitry 1605 is in the “on” state (or active state) and permits current to flow between the terminals 1612 and 1614, the negative pressure source 1604 can receive power from the power source 1602. As a result, delivery of negative pressure wound therapy can be controlled via the play/pause switch.

The switch 1632 (or switch 1634) can be a transistor with lower gate threshold voltage. This can facilitate one or more of reduced power dissipation by the transistor in the event of a short circuit (for instance, as a result of lower “on” resistance of the transistor) or reduced time for the fuse to open (as explained below). For example, the gate threshold voltage of the transistor can be less than or equal to −1.0V, less than or equal to −0.5V, less than or equal to −0.4V, or the like. Additional details of the latching circuitry are disclosed in U.S. Publication No. 2020/0338243, published on Oct. 29, 2020, titled “SAFE OPERATION OF INTEGRATED NEGATIVE PRESSURE WOUND TREATMENT APPARATUSES,” which is incorporated by reference in its entirety.

In some cases, the latching circuitry 1605 may be inadvertently activated. For instance, during manufacturing at least some of the electronic components can be coated with waterproof material. The coating may be cured by being exposed to high temperatures or light (such as, ultraviolet (UV) light). During curing, the switch 1632 may be turned on inadvertently through exposure to one or more of high temperature or light. This may occur as a result of an increased leakage current through the switch 1632, which may cause the capacitor 1636 to charge and turn on the switch 1634.

To prevent inadvertent activation, approaches illustrated in FIGS. 10B-10D may be used. With reference to FIG. 10B, the circuitry 1600B can be similar to the circuitry 1600A except that an activation switch or control 1622 is positioned as shown in the circuitry 1600B. The activation control 1622 can be a pull tab. When the activation control 1622 has been activated (such as, the pull tab has been removed), the LAT_OUT terminal 1614 is connected to the ground. As a result, current supplied by the power source 1602 would flow to the ground rather than to the negative pressure source 1604, thereby preventing activation of the negative pressure source. In this implementation, the activation control 1622 can be activated during manufacturing to prevent inadvertent activation and subsequently deactivated. For instance, the pull tab can be removed during curing and subsequently reattached.

In some instances, the activation control 1622 can be an optical sensor (such as, a photodiode) that turns on in response to being exposed to light. For example, the optical sensor can be turned on by exposure to light during curing. After curing has been completed, the optical sensor would turn off.

With reference to FIG. 10C, the circuitry 1600C can be similar to the circuitry 1600A and 1600B except that a temporary link 1624 (such as, a jumper) to the ground is positioned as shown. The temporary link 1624 can be a pull tab or another conductive component. The temporary link 1624 can be positioned in the circuitry 1600C to prevent inadvertent activation, for instance, during manufacturing. Subsequently, the temporary link 1624 can be removed to permit activation and provision of negative pressure wound therapy.

With reference to the circuitry 1600D illustrated in FIG. 10D, the controller 1550 can detect inadvertent activation and perform appropriate remedial action. The controller 1550 can be control an interface 1628 (which can be a switch, such as a transistor). For instance, the controller 1550 can have an analog output that controls the interface 1628. The controller 1550 can activate the interface 1628 (such as, turn on the transistor), which would cause the line 1626 (connected to the LAT_OUT terminal 1614) to be connected to the ground. As a result, the capacitor 1636 would be discharged causing the switch 1634 to turn off and, in turn, causing the switch 1632 to turn off Effectively, this would cause the latching circuitry to be deactivated. The current supplied by the power source 1602 would flow to the ground rather than to the negative pressure source 1604, thereby preventing activation of the negative pressure source.

During manufacturing, the controller 1550 can be activated (for example, by touching a test point on the circuit board). Activation of the controller 1550 may be performed to verify that the controller is operational. As described herein, the controller 1550 can operate in a low power mode (such as, sleep mode) until one or more of the latching circuitry has been activated and the play/pause button has been activated (such as, pressed). The controller 1550 can monitor the duration of time since it has been activated. The controller can also be connected to the LAT_OUT 1614 terminal. After an expiration of a threshold period of time, the controller 1550 may permit flow of current to the LAT_OUT terminal 1614 by not activating the interface 1628. The period of time can be sufficiently long to accommodate one or more of completion of manufacturing (during which inadvertent activation may occur) and shipping the device to a medical facility for use on a patient. In some cases, the period of time can be 24 hours or less, 36 hours, 48 hours or more, or the like.

Activation of the latching circuitry 1605 prior to the expiration of the period of time would likely be caused by an inadvertent activation. Accordingly, the controller 1550 can activate the interface 1628, which would prevent flow of current from the power source 1602 to the LAT_OUT terminal 1614 and prevent activation of the negative pressure source. In some cases, the circuitry 1600D can include one or more of the activation control 1622 or temporary link 1624.

### Play/Pause Switch

As described herein, the play/pause switch (such as, a button or slide switch) can permit a user to apply or pause application of negative pressure wound therapy. Output from the play/pause switch can be provided to the controller, which can determine whether the play/pause switch has been activated or deactivated. Responsive to the activation of the play/pause switch (such as, press of a button or sliding of the slide switch), the controller can attempt to activate the negative pressure source. When the latching circuitry has been activated such that power is being provided to the negative pressure source (for instance, current from the power source 1602 is being provided), the controller can cause the negative pressure source to be activated and provide negative pressure wound therapy. However, if the latching circuitry has not been activated (for example, because the activation control 1620 has not been activated), the controller would not be able to cause the negative pressure source to be activated since the negative pressure source is isolated from the power source.

In some cases, power to the play/pause switch can be provided from a power source that is not controlled by the latching circuitry. For example, low voltage power to the play/pause switch from one of the battery cells, such by connecting the play/pause switch to the LAT_IN terminal (which can be connected to, for instance, a 3V power source). Activation of the play/pause switch would cause the controller to perform one or more actions associated with attempting activation of the negative pressure source regardless of the activation state of the latching circuitry. This can cause needless consumption of power (for instance, from the battery cell 1502) because responsive to the activation of the play/pause button (such as, a button press or sliding of the slide switch), the controller would attempt, to activate (or deactivate) the negative pressure source even when the latching circuitry has not been activated. In some cases, to conserve power the controller can operate in the low power mode (such as, sleep mode) when the latching circuitry has not been activated. Responsive to receiving an output from the play/pause button, the controller can be configured to 1) switch its operation mode to a higher power mode (such as, wake up from sleep), 2) attempt to activate (or deactivate) the negative pressure source, 3) determine that such attempt has failed due to the latching circuitry not having been activated, and 4) return to operating in the lower power mode. Performing these operations can lead to needless waste of power.

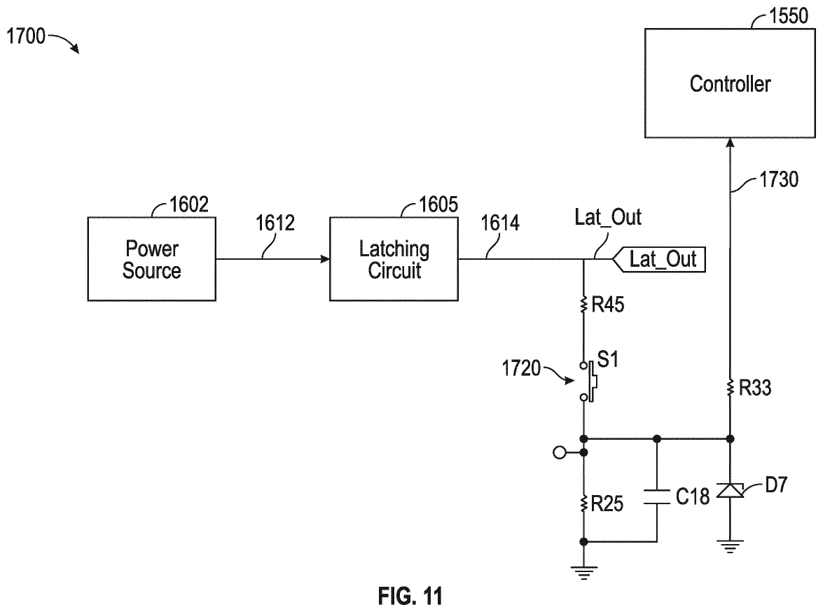

Alternatively, it can be advantageous provide power to the play/pause switch from the power source that is isolated by the latching circuitry. This way, when the latching circuitry has not been activated, the play/pause switch may not provide any output in response to being manipulated (such as, pressed by the user). In some cases, the play/pause switch can be connected to the LAT_OUT terminal 1614 (for instance, 6V). FIG. 11 illustrates a circuit 1700 that embodies such implementation. A play/pause switch 1720 (such as, a button) can be connected to the output of the latching circuitry 1605, which can be LAT_OUT. As shown, a resistor R45 can be interposed between the LAT_OUT terminal 1614 and the play/pause switch 1720. The output 1730 of the play/pause switch 1720 can be provided to the controller 1550. The output 1730 can be a signal corresponding to the output at the LAT_OUT terminal 1614 (such as, 6V). Advantageously, the play/pause switch 1720 may provide output (such as, voltage provided by the LAT_OUT terminal) only when the latching circuit 1605 has been activated, which may be caused by activation of the activation control 1620.

In some cases, the play/pause switch can relay to the controller 1550 one or more outputs beyond the binary “on” and “off.” For example, multiple sequential presses of a button (or sliding of the switch) can cause the electronics to turn off (such as, one or more of cause the latching circuitry to be deactivated, which can be accomplished by discharging the capacitor 1636 as described herein or cause the controller to operate in the low power mode). As another example, multiple different sequential presses of the button (or sliding of the switch) can cause adjustment of one or more parameters of negative pressure wound therapy, such as the negative pressure set point, duty cycle of the negative pressure source, or the like. As yet another example, a short press of the button (or sliding of the switch) can cause negative pressure to be applied for a limited duration of time (such as, to facilitate testing and verification during manufacturing). As yet another example, a long press of the button (or sliding of the switch) can cause the electronics to turn off, for instance, after testing and verification during manufacturing has been completed. The controller can detect one or more of the various different sequences of presses or durations of the one or more presses of the button (or sliding of the switch) and take an appropriate action (or cause an appropriate action to be taken) in response to the detection. Such detection (or any of the other detections disclosed herein) can be performed by the controller executing suitable firmware or software. Advantageously, a variety of multiple user inputs can be provided using the play/pause switch, even in cases where the play/pause switch may be a single control provided to the user.

### Protection Against Excessive Current

With reference to FIG. 9A, the fuses 1582 and 1584 can provide protection against excessive current (or provide overcurrent protection) for the battery cells 1502 and 1504, respectively. While multiple fuses F1-1 to F1-3 (sometimes referred to as group F1-x) and F2-1 to F2-3 (sometimes referred to as group F2-x) are illustrated, in some cases, only one fuse from each for the groups F1-x and F2-x may be populated onto a circuit board represented by the circuit 1505A. As illustrated in FIG. 9A, only fuses F1-1 and F2-1 can be populated onto the circuit board.

The fuses 1582 and 1584 can be configured to provide protection against excessive current. Excessive current can be caused by liquid ingress (such as, liquid aspirated from the wound), which can cause one or more short circuits. For example, the fuse 1582 can protect from excessive current when the battery cell 1502 alone or both battery cells 1582 and 1584 provides power. The fuse 1582 can be configured to open (or blow) when the current in the circuit satisfies a first maximum current threshold. As another example, the fuse 1584 can protect from excessive current when the both battery cells 1502 and 1504 provide power. The fuse 1584 can be configured to open when the current in the circuit satisfies a second maximum current threshold (which may be the same or different as the first maximum current threshold). In some cases, the second maximum current threshold current can correspond to the maximum current threshold of the negative pressure source 1604. In some implementations, the first maximum current threshold current can correspond to the maximum current threshold of the controller 1550.

In some cases, at least one of the fuses 1582 or 1584 can be configured to open when current in the circuit does not satisfy the first or second maximum current threshold. For example, the second maximum current threshold can be about 500 mA (or less or more), which can correspond to the maximum current threshold of the negative pressure source. However, the fuse 1584 can be selected as being configured to open at a current less than the maximum current of the negative pressure source in cases the negative pressure source is being operated discontinuously so that it would likely not consume full power. For instance, the negative pressure source may be activated and deactivated for alternating time durations (such as, pulsed on and off). In such cases, the negative pressure source would likely not draw full power being provided to it. As a result, the fuse 1584 can be selected as being configured to open at a current less than 500 mA. Advantageously, this can promote additional patient protection and safety.

Selecting one or more of fuses 1582 and 1584 can also involve considering temperature. Because the electronics can be supported by a dressing positioned on the patient, allowing the temperature of one or more electronic components to increase as a result of excessive (or nearly excessive) current can cause discomfort to the patient, burn the patient, or otherwise compromise patient comfort or safety as well as cause a fire. To address such risks, one or more of the fuses 1582 or 1584 can be fast acting fuses. Such fuses can have fast response time for opening (for example, about 5 second or less or more). As a result, one or more of the fuses 1582 or 1584 can advantageously act quickly to protect the patient against excessive current and temperature. It can be advantageous to select a fuse with lower resistance to facilitate conserving power source capacity. For instance, resistance of the fuse can be about 200 mOhm (or less or more).

One or more of the fuses 1582 or 1584 can be one time fuses that would need to be replaced after providing overcurrent protection or resettable fuses (or positive temperature coefficient, PTC) that can provide overcurrent protection for a number of times before needing replacement.

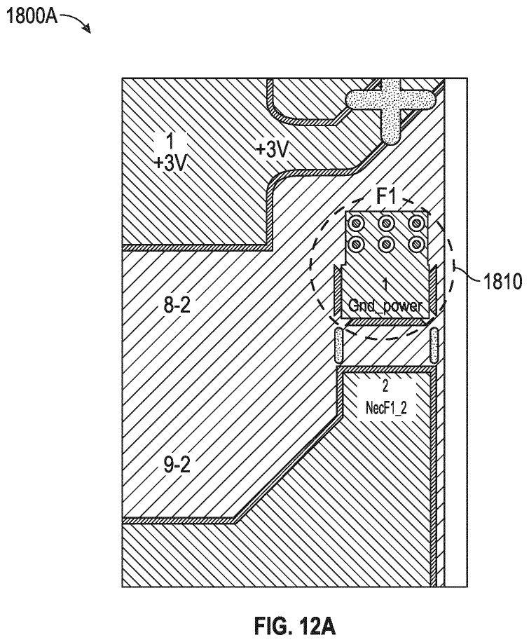

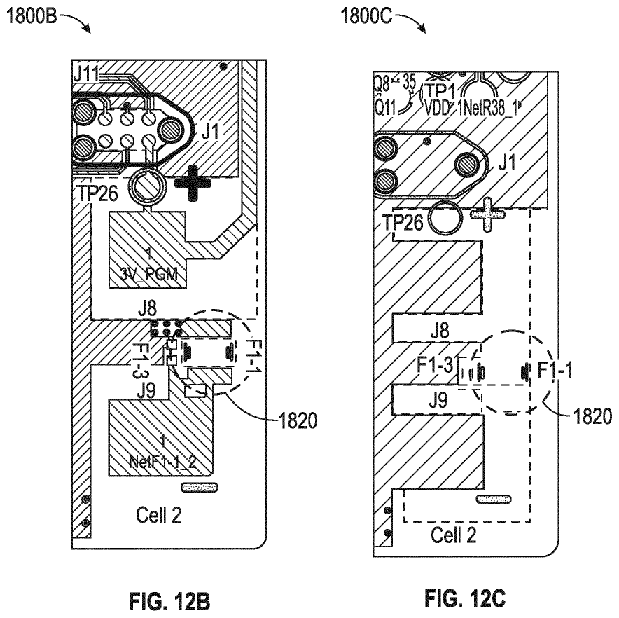

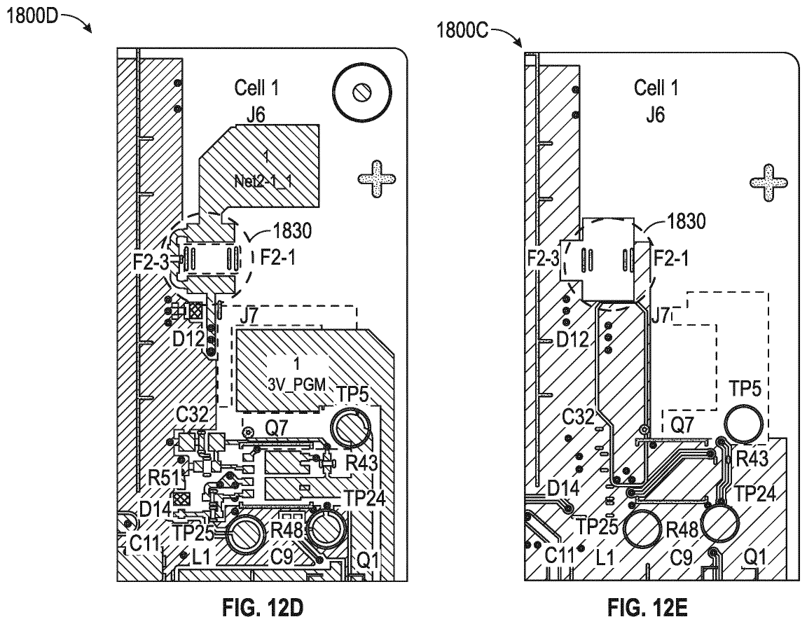

FIG. 12A illustrates a layout 1800A of a circuit board. Area 1810 can illustrate location of where a fuse F1 would be positioned (this fuse has been removed from the circuit board to facilitate the explanation). As is shown, the area 1810 includes conductive metal (such as, copper) surrounding the fuse F1. The conductive metal can be part of a ground plane or a power plane of the circuit board. Such design can be disadvantageous since the conductive metal can provide a heat sink for the fuse F1. In some cases, it can be important to allow the fuse to “warm up” so that it quickly blows when excessive current is being conducted. Being surrounded by conductive metal can prevent the fuse F1 from warming up. As a result, there can be an undesirable delay before the fuse F1 reaches the temperature needed for blowing.

Thermal isolation can solve these problems. FIGS. 12B and 12C illustrate layouts 1800B and 1800C of a circuit board (which can be a double sided or two layer printed circuit board) in which thermal isolation for the fuse F1-1 (as well as the other fuses F1-2 and F1-3) has been implemented. Layout 1800B in FIG. 12B can illustrate the top layer of the circuit board. One or more electronic components can be positioned on the top layer. As is illustrated by the blank space (or void) in the area 1820, conductive metal (such as, copper) has been removed from the area on the top layer where the fuse F1-1 (as well as the other fuses F1-2 and F1-3) would be positioned. Conductive metal can also be removed from the area surrounding the fuse F1-1 (as well as the other fuses F1-2 and F1-3). Such removal of conductive metal can prevent formation of a heat sink on the top layer for the fuse 1582.

Layout 1800C in FIG. 12C can illustrate the bottom layer of the circuit board. The bottom layer can be primarily used for wiring and soldering. As is illustrated by the blank space (or void) in the area 1820, conductive metal (such as, copper) has been removed from the area on the bottom layer where the fuse F1-1 (as well as the other fuses F1-2 and F1-3) would be positioned. Conductive metal can also be removed from the area surrounding the fuse F1-1 (as well as the other fuses F1-2 and F1-3). Such removal of conductive metal can prevent formation of a heat sink on the bottom layer for the fuse 1582.

FIGS. 12D and 12E illustrate layouts 1800D and 1800E of the circuit board in which thermal isolation for the fuse F2-1 (as well as the other fuses F2-2 and F2-3) has been implemented. Layout 1800D in FIG. 12D can illustrate the top layer of the circuit board. As is illustrated by the blank space (or void) in the area 1830, conductive metal (such as, copper) has been removed from the area on the top layer where the fuse F2-1 (as well as the other fuses F2-2 and F2-3) would be positioned. Conductive metal can also be removed from the area surrounding the fuse F2-1 (as well as the other fuses F2-2 and F2-3). Such removal of conductive metal can prevent formation of a heat sink on the top layer for the fuse 1584.

Layout 1800E in FIG. 12E can illustrate the bottom layer of the circuit board. As is illustrated by the blank space (or void) in the area 1830, conductive metal (such as, copper) has been removed from the area on the bottom layer where the fuse F2-1 (as well as the other fuses F2-2 and F2-3) would be positioned. Conductive metal can also be removed from the area surrounding the fuse F2-1. Such removal of conductive metal can prevent formation of a heat sink on the bottom layer for the fuse 1584.

Removal of the conductive metal can provide clearance around one or more of the fuses 1582 or 1584. For instance, the clearance can be about 2 to 3 mm (or less or more). In addition to providing thermal isolation, the clearance can avoid creating a short circuit and blowing the one or more of the fuses due to bending or cracking of the printed circuit board (which can be flexible or substantially flexible).

In some cases, the circuit board can be single layer printed circuit board or a printed circuit board with more than two layers. Similar approaches for thermal isolation can be implemented with such circuit boards.

In some implementations, one of more of the fuses 1582 or 1584 can be replaced with a current limiting load switch (such as, a Texas Instrument TPS255xx series switch). A current limiting load switch can have an adjustable current limit and can be configured to open at a desired maximum current threshold. The current limiting load switch can have low resistance (which can be advantageous for conserving power source capacity). For example, the resistance can be about 85 mOhm (or less or more), which may be lower than the resistance of a fuse. The current limiting load switch can open the circuit quickly (that is, have short latch-off time, such as about 10 msec or less), which may be faster than the fuse.

As described above, the switch 1632 (or switch 1634) can be a FET with lower gate threshold voltage. This can reduce the time for one or more of the fuses 1582 or 1584 to open because such switch can conduct increased current (for instance, as a result of lower impedance of the FET). As result, this can permit the fuse to react quickly to excessive current.

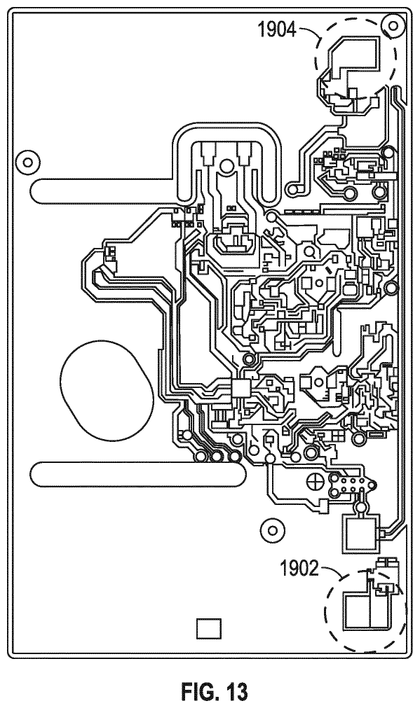

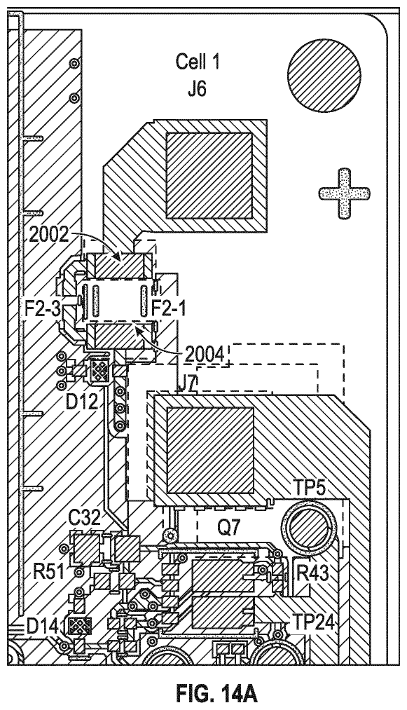

In some cases, not all portions of conductive traces or tracks in the circuit board may be protected by the one or more fuses 1582 or 1584. Such portions can be referred to as unfused tracks. Because the circuit board may be flexible or substantially flexible, it can be advantageous to increase clearance (or separation) between the one or more unfused tracks and conductive components proximal to such one or more tracks. With reference to FIG. 13, area 1902 illustrates the terminal 1512 of the battery cell 1502. With reference to FIG. 9A, this terminal is positioned before the fuse 1582 and, as a result, forms an unfused track. It can be advantageous to increase the clearance between the terminal 1512 and a proximal conductive component (such as, a ground plane). The increased clearance can be at least twice the thickness of the printed circuit board (or larger). For example, the clearance can be about 2 to 3 mm (or less or more).

With reference to FIG. 13, area 1904 illustrates the terminal 1518 of the battery cell 1504. With reference to FIG. 9A, this terminal is positioned before the fuse 1584 and, as a result, forms an unfused track. It can be advantageous to increase the clearance between the terminal 1518 and a proximal conductive component (such as, the power plane). The increased clearance can be at least twice the thickness of the printed circuit board (or larger). For example, the clearance can be about 2 to 3 mm (or less or more).

Increasing the clearance can advantageously prevent shorting of the terminals of the one or more battery cells and can improve electrical isolation. One or more of the battery cells may reach a high temperature (such as, about 90° C. or more or less) when shorted, which may cause discomfort to the patient, burn the patient, or start a fire. Increasing the clearance can promote patient comfort and safety.

With reference to FIG. 9A, groups of fuses F1-x and F2-x are illustrated. As described herein, only one of the fuses in each of the groups may be populated onto the circuit board. Because of miniaturization of the circuit board, the footprints for the fuses F1-1, F1-2, and F1-3 in the group F1-x and three fuses F2-1, F2-2, and F2-3 in the group F2-x may be positioned close together. As a result, there may be a risk of creating a short circuit with the exposed footprints of unpopulated fuses, particularly when the circuit board is flexed. For instance, the fuses may be positioned on the circuit board using surface mount technology (SMT). While SMT can utilize solder balls to mount the fuses onto the circuit board, this can create a risk of splashing, residue, whiskers, or the like, which may create one or more short circuits.

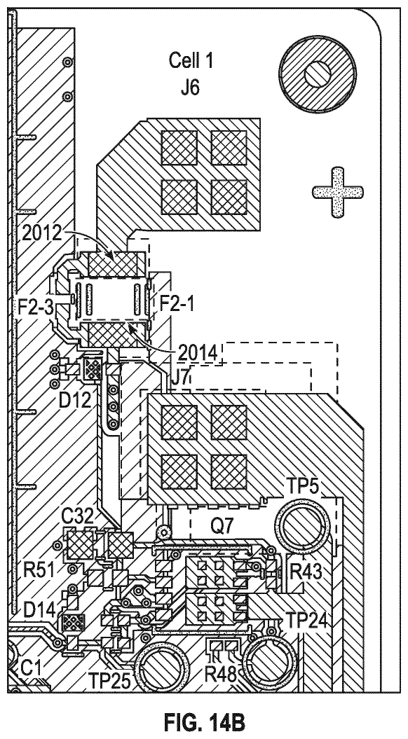

To solve these problems, painting with a solder mask can be utilized to prevent the formation of electrical connections with the exposed terminals for the unpopulated fuses. An area not painted with the solder mask can correspond to a conductive area in which one or more electrical connections would be formed. With reference to FIG. 14A that illustrates forming electrical connections for the fuse F2-1, areas 2002 and 2004 may not be covered with the solder mask when the circuit board is being manufactured. The manufacturing process can have sufficient precision and tolerance to correctly position the solder mask while excluding the areas as small as 2002 and 2004. Areas 2002 and 2004 can correspond to the locations of the terminals or pads of the fuse F2-1. With reference to FIG. 14B, the manufacturing process can subsequently form solder paste in the areas 2002 and 2004 where the solder mask has not been positioned. Such regions of solder paste are illustrated as 2012 and 2014. Conductive pads (which can be formed, for example, from gold) may have been created in the areas 2002 and 2004 subsequently to painting with the solder mask (as illustrated in FIG. 14A) but prior to the formation of the solder paste. As a result, electrical contacts for the fuse F2-1 can be formed without the risk of creating one or more short circuits with the exposed terminals of the surrounding fuses F2-2 and F2-3. Similar approaches can be used for forming electrical connections for the fuse F1-x (such as, the fuse F1-1). Using the solder mask can facilitate precise control of the placement of conductive components and avoid creating short circuits.

### Liquid Ingress Protection

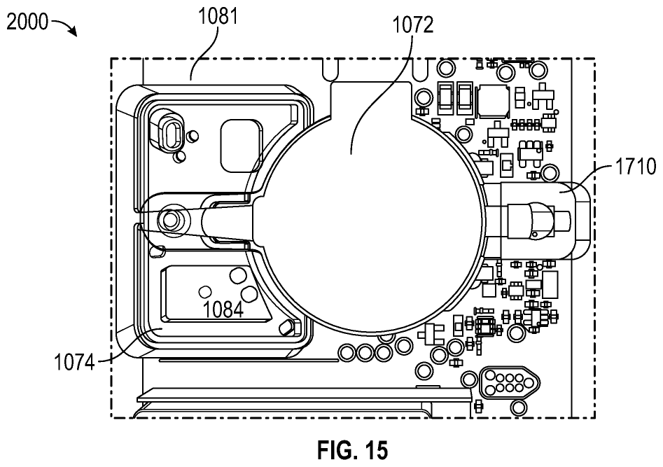

It may be possible for liquid to enter an electronics assembly of the TNP system (such as, any of the assemblies 400, 500, or 1500). This may be particularly likely in cases where the electronic assembly is positioned on the dressing that is placed on the patient, such as described herein. For instance, wound exudate absorbed by the dressing may penetrate the electronics assembly even in cases the assembly is sealed. As another example, condensation may form. As yet another example, the patient may expose the electronics to liquid (such as, by taking a shower when the wound dressing is positioned on the patient). With reference to FIG. 15, liquids may enter an electronics assembly 2000 through the pump inlet protection mechanism 1710 (for instance, because the negative pressure source can aspirate fluids through the inlet covered by the inlet protection mechanism 1710). As another example, with reference to FIG. 15, liquids may enter through the pump exhaust mechanism 1074 (for instance, through the one or more vents 1084). Because wound exudate, other bodily fluids (such as, blood), or medicinal fluids that may be introduced into the wound can be conductive, coming into contact with the electronics can undesirably interfere with the operation of the TNP system and may cause discomfort or injury to the patient. To ensure patient comfort, safety, and correct provision of therapy, it can be advantageous to detect ingress of liquids and take one or more remedial actions responsive to the detection. The one or more remedial actions can include at least one of providing an indication (such as, via the one or more indicators described herein), pausing or stopping the negative pressure source, deactivating the TNP system (such as, temporarily or permanently), or the like. For example, the TNP system may prevent a user from reactivating the application of negative pressure during a first time period (e.g., based on the time required to clear an error caused by the liquid ingress) and allow the user to reactivate the application of negative pressure after expiration of the first time period. Further, the TNP system may permanently disable the reactivation of the application of negative pressure.

With reference to FIG. 15, a pressure sensor (such as, the pressure sensor 1091) can be positioned to measure pressure in the fluid flow path connecting the negative pressure source 1072 to the wound. The pressure sensor 1091 can be positioned in or proximal to the pump inlet protection mechanism 1710. In addition to measuring pressure, the pressure sensor 1091 can measure temperature, such as the internal temperature of the TNP system or of one or more electronic components. Another pressure sensor (such as, the pressure sensor 1092) can measure pressure of the surrounding environment. In addition to measuring pressure, the pressure sensor 1092 can measure temperature, such as the external temperature. The pressure sensor 1092 can be positioned in or proximal to the pump exhaust mechanism 1074 (which can be in fluid communication with the surrounding environment). To facilitate communication with the surrounding environment, there can be one or more vents 1084 in the pump exhaust mechanism 1074, as shown in FIG. 15. Additionally, there can be or more vents in a portion of the circuit board (such as, the circuit board 1081) proximate to the one or more vents 1084.

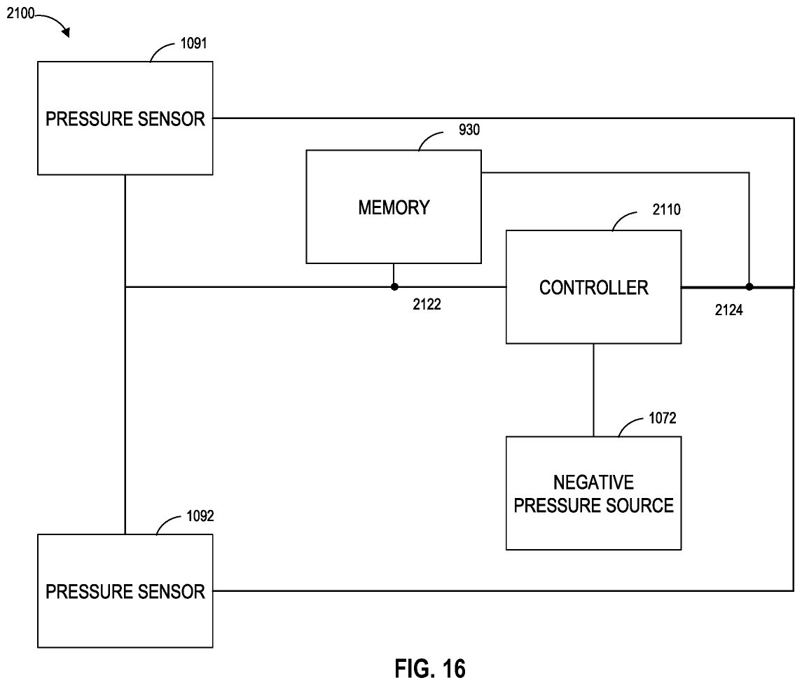

FIG. 16 illustrates a block diagram 2100 of the electronics of a TNP system (which can be any of the systems described herein). The electronics can be positioned on the wound dressing, as described herein. The electronics can include an internal pressure sensor 1091, an external pressure sensor 1092, a negative pressure source 1072, a controller 2110 (which can be any of the controllers described herein, such as the controller 1550), digital signal lines or traces (hereinafter “lines”) 2122 and 2124, and a memory 930. The lines 2122 and 2124 can support a digital communications protocol between the controller 2110 and one or more of the memory 930, pressure sensor 1091, or pressure sensor 1092. The digital communications protocol can be I²C, PMBus, SMBus, SPI, USB, UART, IEEE1394 (or Firewire), CAN, or the like. For example, the lines 2122 and 2124 can be serial data (SDA) and serial clock (SCL) lines used by the I²C protocol. The internal pressure sensor 1091 and/or the external pressure sensor 1092 may measure pressure in the fluid flow path. One or more of the lines 2122 and 2124 may be electrically connected to the internal pressure sensor 1091 and/or the external pressure sensor 1092. For example, at least portions of one or more of the lines 2122 and 2124 may not be coated with waterproof material. This may be due to exposing one or more testing points used during manufacturing (such as, for verification). Alternatively or additionally, portions of the lines 2122 and 2124 (or any other conductive lines or traces) may be exposed in order to generate a conductive path for detecting the occurrence of liquid ingress. For instance, protective rings for detecting liquid ingress can be created. Further or different areas of the TNP system may be exposed in order to detect liquid ingress. As described herein, the TNP system may detect liquid ingress using digital signal traces (e.g., by detecting a short circuit across lines) and/or analog signal traces (e.g., by detecting a short circuit to ground).

The pressure sensors 1091, 1092 and the controller 2110 can by supported by a circuit board, such as the circuit board 1081. For example, the lines 2122 and 2124 may be electrical traces on or within the circuit board 1081 connecting one or more of the pressure sensors 1091, 1092, and the controller 2110. The circuit board 1081 can support one or more electrical traces to transmit one or more digital signals and one or more electrical traces to transmit one or more analog signals.

Liquid ingress detection can be performed by detecting a degradation (such as, any one or more of attenuation, interruption, diminishing, change, or reduction) of digital signals. For example, liquid ingress detection can be performed by detecting a short between the lines 2122 and 2124 (which may correspond to SDA and SCL lines of I²C protocol). The lines 2122 and 2124 may electrically connect the controller 2100 to one or more of the pressure sensors 1091, 1092. During normal operation, data and clock can be transmitted across the lines 2122 and 2124. As a result of liquid ingress, the lines 2122 and 2124 may be shorted together or one or more of the lines 2122 or 2124 may be shorted to the ground or power source. For instance, a pull up resistor positioned between the power source and one of the lines 2122 or 2124 may be bypassed as a result of a short circuit created by the liquid. This can cause degradation of one or more signal transmitted across one or more of the lines 2122 or 2124. For example, the introduction of a fluid may change the impedance and/or capacitance of the lines 2122 and 2124. As a result, one or more of the voltage, current, or timing parameters of the transmitted signals may no longer comply with the I²C protocol.

Such degradation can be detected by the controller 2110. For instance, the controller 2110 may detect one or more errors signaled by the I²C interface of the controller. As another example, the controller 2110 may be unable to read pressure from one or more of the pressure sensors 1091 or 1092 or may read pressure that is out of range of acceptable pressures. In response to the detection of liquid ingress, the controller 2110 can take on or more remedial actions. These can include permanently or temporarily disabling the negative pressure source, permanently or temporarily disabling the TNP system, or providing an indication of liquid ingress.

As described herein, at least some portions of the lines 2122 and 2124 may be left exposed (or without coating). Liquid ingress may cause formation of an electrical short (e.g., connection) between the line 2122 and the line 2124 (or between one or more of the lines 2122 or 2124 and the ground and/or power source). This can occur due to liquid being electrically conductive (for example, wound exudate). The electrical short may be based on the current flowing along an unintended path with reduced impedance. As a result, excessive current to flow in the electronics. The excessive current flowing through the TNP system can cause a rapid increase in the temperature, which may cause a burn, a fire, etc.

Similarly, a short circuit between the lines 2122 and 2124 or on one or more of the lines 2122 or 2124 may cause the memory 930 to become inaccessible. For example, the controller 2110 may attempt to access the memory 930 (e.g., to read from or write to the memory 930) and, in response to being unable to access the memory, may determine that the memory 930 is inaccessible. In some cases, the controller 2110 may determine that the memory 930 is inaccessible based on not receiving a response to a request to access the memory 930 during a threshold period of time.

As described herein, additionally or alternatively to detecting liquid ingress based on detecting degradation of digital signals, liquid ingress detection can be performed by detecting degradation of analog signals. For example, the controller 2110 can detect a change in a voltage or the voltage reaching a particular threshold (e.g., an out-of-range voltage). The controller 2110 may use the analog signal traces in order to detect the degradation. The controller 2110 may detect the voltage at an analog to digital convertor, a comparator, a feedback line, etc. As is described herein, the degradation can be caused by a short circuit (such as, a short circuit to the ground, power source, or between two or more traces

Based on detecting the degradation of one or more digital or analog signals, the electronics (such as, the controller 2110) can mitigate the effects of the liquid ingress (e.g., to protect the TNP system). For example, responsive to the detection of the degradation, the controller 2110 can temporarily or permanently deactivate provision of negative pressure to a wound. Alternatively or additionally, the controller 2110 may mitigate the effects of the liquid ingress by generating an indication (such as, an alarm), blowing a fuse, opening a fuse, opening a switch, or opening a relay.

As described herein, the electronics can include one or more batteries. In response to the detection of liquid ingress, the controller 2110 may deplete (or discharge) the one or more batteries in order to promote patient safety and/or for environmental reasons (such as, for waste management without the risk of fire or explosion). For instance, U.S. regulations provide that, generally, a lithium battery cell is considered to be discharged once its voltage reaches 2V or less under a current of C/100 (where C is the rated capacity of the battery in ampere-hours).

The circuitry for depleting the one or more batteries may be a smart discharge circuitry utilizing a load to control the temperature during the discharge of the one or more batteries. For instance, the one or more batteries can become hot as a result of the draining, which may cause discomfort or injury to the patient. The circuitry for depleting the one or more batteries may be coated in order to protect the circuitry from liquid ingress. The circuitry for depleting the one or more batteries can include switch (such as, a transistor) and a load (e.g., a resistor or a resistor network that includes a plurality of resistors with different resistances) connected to the switch. Different resistors in the network can be used to control the temperature during the discharge. The load can include a conductive plane (e.g., a copper plane) of the circuit board. The controller 2110 can activate the switch and drain the one or more batteries through the load. The controller 2110 can track the temperature of the TNP system (for instance, the temperature of the one or more batteries) and deplete the one or more batteries based on the temperature. The controller 2110 may measure a feedback current and deplete the one or more batteries based on the feedback current. For example, the feedback current may be a proxy for the temperature. In response to detecting that the feedback current and/or the temperature do not satisfy (such as, are below) a particular temperature threshold indicative of a high temperature, the controller 2110 can activate the switch to initiate a discharge of the one or more batteries. While tracking of the temperature for the discharge can be performed based on the reading by one or more of the pressure sensors 1091 or 1092, it may be advantageous to track the temperature with a separate temperature sensor (in case the pressure sensor 1091 or 1092 has been compromised by liquid ingress).

A plurality of temperature thresholds may be used to facilitate the discharge through a resistor network. For example, responsive to the temperature not satisfying (such as, being below) a first temperature threshold, discharging through a first higher resistance resistor in the network can be performed. Responsive to the temperature satisfying the first temperature threshold but not satisfying a second temperature threshold associated with a higher temperature than the first temperature threshold, discharging through a second resistor in the network (with lower resistance than the first resistor) can be performed.

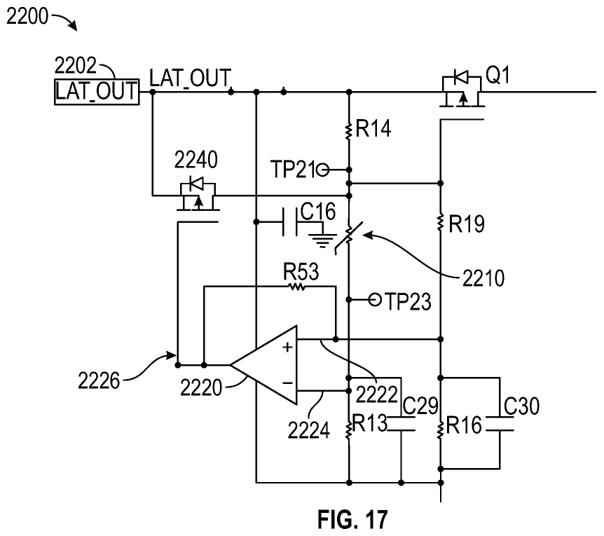

The following is an example of detecting liquid ingress using analog signal traces. FIG. 17 illustrates a diagram of circuitry 2200 configured to perform over-temperature detection in a TNP system (which can be any of the TNP systems described herein). A temperature sensor 2210 can monitor temperature of one or more components of the TNP system. For example, the temperature sensor 2210 can monitor temperature of a negative pressure source or a boost converter (or boost regulator). The boost converter can increase the power provided by a power source (such as, the power source 1068) to a power level adequate for powering the negative pressure source (such as, the negative pressure source 1072). The temperature sensor 2210 can be a thermistor. Temperature monitored by the temperature sensor 2210 (which can be output as a voltage signal) can be provided to a comparator 2220 (for instance, as an input 2224). The comparator 2220 can be an operational amplifier. The other input 2222 to the comparator 2220 can correspond to a temperature threshold, such as a maximum temperature threshold at or below which the TNP system is designed to operate safely and effectively. For example, the maximum temperature threshold can be about 40 degrees Celsius or less or more, 41 degrees Celsius or less or more, 42 degrees Celsius or less or more, 48 degrees Celsius or less or more, 50 degrees Celsius or less or more, or the like. The output 2226 of the comparator 2220 can be indicative of whether the temperature monitored by the temperature sensor 2210 satisfies the temperature threshold. The output 2226 can be used to control a switch 2240, such as a transistor (in which case, the output 2226 can control the gate of the transistor). The output of the switch 2240 can be used to override the output of a latching circuitry, which can facilitate provision of power from the power source to one or more of the other electronics components, such as to the boost converter or negative pressure source 1072. The latching circuitry can be activated by removal of the pull tab, as described herein. In case the temperature monitored by the temperature sensor 2210 satisfies the temperature threshold, the switch 2240 would be turned on. This can cause the output of the latching circuitry to be overridden and the negative pressure source to be deactivated (for instance, due to the deactivation of the boost converter). For example, activation of the switch 2240 can cause the output 2202 to be a low voltage (such as, the ground), which can provide an indication to the controller to deactivate the negative pressure source. In some cases, overriding the output 2202 can cause the boost converter to be deactivated. Additional details of over-temperature detection are described in International Patent Publication No. WO2022/073762 titled “TEMPERATURE MONITORING AND CONTROL FOR NEGATIVE PRESSURE WOUND THERAPY SYSTEMS,” which is incorporated by reference herein in its entirety.

As a result of liquid ingress, the terminals of the temperature sensor 2210 may be shorted. As another example, the inputs 2222 and 2224 to the comparator 2220 may be shorted. Due to such one or more short circuits caused by the liquid ingress, the switch 2240 can be activated, thus indicating over-temperature. The controller 2110 may verify whether the detected over-temperature is incorrect by analyzing temperature measured by another temperature sensor, such temperature measured by one or more of the pressure sensors 1091 or 1092. The temperature measured by the temperature sensor 2210 and the temperature(s) measured by the one or more pressure sensors 1091 or 1092 should be correlated. The controller 2110 can determine that the over-temperature detection is incorrect based on temperature measured by another temperature sensor not satisfying or being close (such as, within 5% or less or more, 10% or less or more, 20% or less or more) to the maximum temperature threshold. Based on the verification of the temperature measured by another temperature sensor, the controller can determine that over-temperature was detected incorrectly and conclude that this error was caused by liquid ingress.

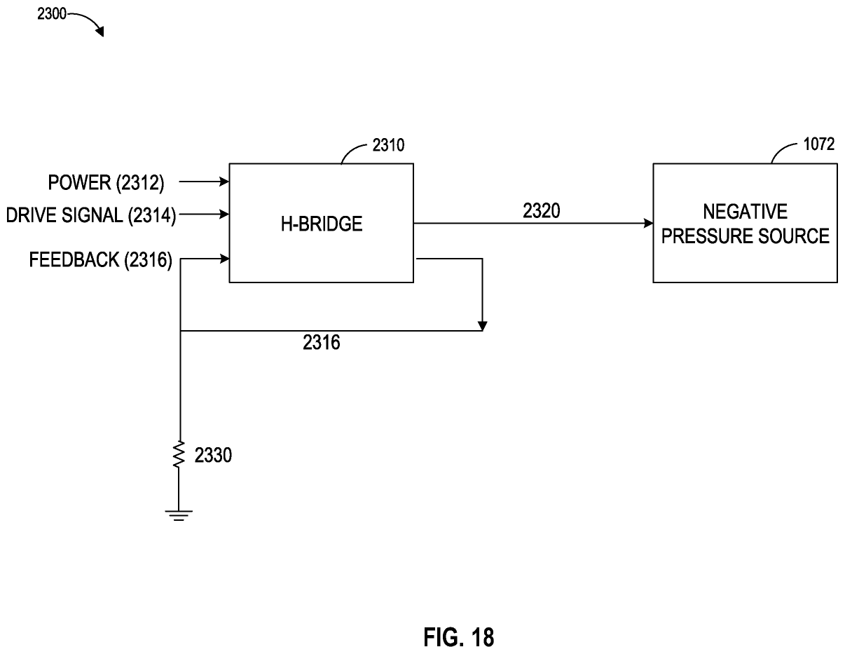

The following is another example of detecting liquid ingress using analog signal traces. FIG. 18 illustrates a block diagram 2300 of circuitry configured to drive a negative pressure source (such as, the pump 1072). In some cases, the negative pressure source can include a piezoelectric pump (such as, a pump operated by a piezoelectric actuator or transducer). Such pump can be driven by an H-bridge circuitry 2310, which can switch polarity of voltage applied to the negative pressure source. The H-bridge circuitry 2310 can receive power 2312 (for example, from the boost converter) and a drive signal 2314 (for example, from the controller 2110). The drive signal 2314 can be a periodic signal at a desired duty cycle, for instance, a square wave or a sinusoidal wave signal. The H-bridge circuitry 2310 can generate an output drive signal for driving the negative pressure source. The output drive signal can be an electrical signal scaled by the received power 2312 (such as, at or substantially at the voltage level of the received power 2312) with a duty cycle corresponding to the drive signal 2314.

A feedback signal (or feedback current) 2316 can be used to monitor the current 2320 provided to the negative pressure source. The feedback current can be used, for instance, to determine the efficiency of the negative pressure source, to protect the negative pressure source from unsafe current, or the like. The feedback current 2316 can be measured across a resistor 2330 (or a feedback resistor).

Liquid ingress within the TNP system may short the terminals of the resistor 2330. For example, liquid ingress may short the feedback resistor 2330 to ground. This can cause the H-bridge circuitry 2310 to detect an error. In turn, the controller 2110 can detect the error and, as a result, determine liquid ingress. In some cases, the controller 2110 can directly detect the error across the feedback resistor 2330.

In some instances, liquid ingress detection based on degradation of one or more digital signals may be faster than detection based on degradation of one or more analog signals. The controller 2110 can directly receive or process one or more digital signals. For instance, the controller 2110 can process one or more digital signals by executing firmware or software, which can determine the source of an error due to the degradation in one or more digital signals and detect liquid ingress.

Alternatively or additionally, the TNP system may also include one or more electronic components (e.g., a humidity sensor, an electronic fuse, etc.) to detect the liquid ingress. The controller 2110 can detect liquid ingress based on a signal received from the one or more electronic components. For example, the controller 210 can detect liquid ingress based on receiving a signal from the humidity sensor identifying a change in humidity (e.g., an increase in humidity) and/or the humidity reaching a particular threshold (e.g., a relative humidity of 75%). The humidity sensor may measure an absolute humidity, a relative humidity, or a specific humidity. Further, the controller 2110 can detect liquid ingress based on a receiving a signal from an electronic fuse (or eFuse) identifying an overcurrent or overvoltage condition.

### CONCLUSION

Medical devices, including negative pressure wound therapy devices, can be held to heightened safety standards. For example, IEC 60601-1 technical standard for the safety of performance of medical electrical equipment provides that a type BF or CF medical device (that is electrically connected to the patient, but not directly to the heart) must be single fault safe. This means that such device must remain free of unacceptable risk during its expected service life under single fault conditions (a condition in which a single means for reducing a risk is defective, or a single abnormal condition is present).

The approaches described herein can provide mitigation against the risk of one or more malfunctions of the electronics. The one or more malfunctions can include flow reverse current, flow of excessive current, or inadvertent activation. The approaches described herein can provide protection against a single fault (or higher protection against more than one fault). Advantageously, mitigation against the risk of burning or otherwise causing discomfort to the patient or the risk of fire can be provided.

Disclosed approaches for detecting degradation of digital or analog signals can facilitate early sensing of liquid ingress, condensation, or ingress of other conductive materials and can be applicable to any wearable medical device. This can facilitate early risk control of the medical device safety and patient safety, including early alarming generation, prevention of patient discomfort or injury, prevention of fire, or the like. As described herein, portions of one or more conductive lines or traces may be exposed (such as, not coated) to create protective regions or areas (such as, protective rings) for liquid ingress detection. The disclosed approaches can be inexpensive to implement as they may not require any additional electronic components or may require very few additional electronic components.

### Other Variations

While certain embodiments described herein relate to integrated negative pressure wound therapy systems in which the negative pressure source is supported by the dressing, systems and methods described herein are applicable to any negative pressure wound therapy system or medical system, particularly to systems being positioned on (or worn by) the patient. For example, systems and methods for controlling operation described herein can be used in fluid-proof (such as, water-proof) negative pressure wound therapy systems or medical systems. Such systems can be configured with the negative pressure source and/or electronics being external to the wound dressing, such as with the negative pressure source and/or electronics being positioned in a fluid proof enclosure. Additionally, such systems can be configured to be used within ultrasound delivery devices, negative pressure devices powered by an external power supply, negative pressure devices with a separate pump, and medical devices generally.

Any of the embodiments disclosed herein can be used with one or more features disclosed in U.S. Pat. No. 7,779,625, titled “DEVICE AND METHOD FOR WOUND THERAPY,” issued Aug. 24, 2010; U.S. Pat. No. 7,964,766, titled “WOUND CLEANSING APPARATUS IN SITU,” issued on Jun. 21, 2011; U.S. Pat. No. 8,235,955, titled “WOUND TREATMENT APPARATUS AND METHOD,” issued on Aug. 7, 2012; U.S. Pat. No. 7,753,894, titled “WOUND CLEANSING APPARATUS WITH STRESS,” issued Jul. 13, 2010; U.S. Pat. No. 8,764,732, titled “WOUND DRESSING,” issued Jul. 1, 2014; U.S. Pat. No. 8,808,274, titled “WOUND DRESSING,” issued Aug. 19, 2014; U.S. Pat. No. 9,061,095, titled “WOUND DRESSING AND METHOD OF USE,” issued Jun. 23, 2015; U.S. Pat. No. 10,076,449, issued Sep. 18, 2018, titled “WOUND DRESSING AND METHOD OF TREATMENT”; U.S. patent application Ser. No. 14/418,908, filed Jan. 30, 2015, published as U.S. Publication No. 2015/0190286, published Jul. 9, 2015, titled “WOUND DRESSING AND METHOD OF TREATMENT”; U.S. Pat. No. 10,231,878, titled “TISSUE HEALING,” issued Mar. 19, 2019; PCT International Application PCT/GB2012/000587, titled “WOUND DRESSING AND METHOD OF TREATMENT” and filed on Jul. 12, 2012; International Application No. PCT/IB2013/001469, filed May 22, 2013, titled “APPARATUSES AND METHODS FOR NEGATIVE PRESSURE WOUND THERAPY”; PCT International Application No. PCT/IB2013/002102, filed Jul. 31, 2013, titled “WOUND DRESSING AND METHOD OF TREATMENT”; PCT International Application No. PCT/IB2013/002060, filed Jul. 31, 2013, titled “WOUND DRESSING AND METHOD OF TREATMENT”; PCT International Application No. PCT/IB2013/00084, filed Mar. 12, 2013, titled “REDUCED PRESSURE APPARATUS AND METHODS”; International Application No. PCT/EP2016/059329, filed Apr. 26, 2016, titled “REDUCED PRESSURE APPARATUSES”; PCT International Application No. PCT/EP2017/059883, filed Apr. 26, 2017, titled “WOUND DRESSINGS AND METHODS OF USE WITH INTEGRATED NEGATIVE PRESSURE SOURCE HAVING A FLUID INGRESS INHIBITION COMPONENT”; PCT International Application No. PCT/EP2017/055225, filed Mar. 6, 2017, titled “WOUND TREATMENT APPARATUSES AND METHODS WITH NEGATIVE PRESSURE SOURCE INTEGRATED INTO WOUND DRESSING”; PCT International Application No. PCT/EP2018/074694, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/074701, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/079345, filed Oct. 25, 2018, titled “NEGATIVE PRESSURE WOUND TREATMENT APPARATUSES AND METHODS WITH INTEGRATED ELECTRONICS”; PCT International Application No. PCT/EP2018/079745, filed Oct. 30, 2018, titled “SAFE OPERATION OF INTEGRATED NEGATIVE PRESSURE WOUND TREATMENT APPARATUSES”; each of which is incorporated by reference herein in its entirety.

Although certain embodiments described herein relate to wound dressings, systems and methods disclosed herein are not limited to wound dressings or medical applications. Systems and methods disclosed herein are generally applicable to electronic devices in general, such as electronic devices that can be worn by or applied to a user.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure.

The various components illustrated in the figures or described herein may be implemented as software or firmware on a processor, controller, ASIC, FPGA, or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A negative pressure wound therapy system comprising:
a negative pressure source configured to provide negative pressure to a wound covered by a wound dressing and to aspirate fluid from the wound; and
a circuit board supporting a plurality of electronic components, the plurality of electronic components comprising control circuitry configured to control operation of the negative pressure source, the circuit board supporting a plurality of traces electrically connecting the plurality of electronic components, the plurality of traces comprising a first set of traces configured to transmit one or more digital signals and a second set of traces configured to transmit one or more analog signals,
the control circuitry further configured to:
detect a degradation of an electrical signal on at least one trace of the first or second set of traces, the degradation caused by one or more of an ingress of liquid onto the circuit board or condensation; and
responsive to detection of the degradation of the electrical signal caused by one or more of the ingress of liquid or condensation, temporarily or permanently deactivate provision of negative pressure to the wound.

2. The system of claim 1, further comprising the wound dressing, wherein at least one of the negative pressure source or the circuit board is at least partially supported by the wound dressing.

3. The system of claim 1, wherein the circuit board supports the negative pressure source.

4. The system of claim 1, wherein the ingress of liquid occurs as a result of the negative pressure source aspirating fluid from the wound.

5. The system of claim 1, wherein the degradation of the electrical signal is caused by a short circuit as a result of the one or more of the ingress of liquid or condensation.

6. The system of claim 4, wherein the degradation of the electrical signal is caused by a short circuit between two traces from the first set of traces or between a trace from the first set of traces and ground or power.

7. The system of claim 6, wherein the two traces from the first set of traces comprise portions not coated with waterproof material or the trace from the first set of traces comprises a portion not coated with waterproof material, and wherein the short circuit is formed due to liquid coming into contact with the portions not coated with waterproof material or the portion not coated with waterproof material.

8. The system of claim 1, wherein a trace from the second set of traces corresponds to a feedback line of the negative pressure source, and wherein the control circuitry is configured to detect the degradation of the electrical signal based at least in part on a short across the feedback line.

9. The system of claim 1, wherein a trace from the second set of traces is part of a circuitry configured to detect excessive temperature, and wherein the control circuitry is configured to detect the degradation of the electrical signal by determining that the circuitry configured to detect excessive temperature has made an incorrect detection of excessive temperature.

10. The system of claim 9, wherein the control circuitry is configured to determine that the circuitry configured to detect excessive temperature has made the incorrect detection of excessive temperature based on processing temperature detected by an additional temperature sensor.

11. The system of claim 1, further comprising at least one battery, wherein the control circuitry is further configured to drain the at least one battery responsive to the detection of the degradation of the electrical signal.

12. The system of claim 11, wherein the plurality of electronic components further comprise a resistor network and a switch, and wherein the control circuitry is configured to open the switch and drain the at least one battery through the resistor network.

13. The system of claim 11, wherein the plurality of electronic components further comprise a conductive plane and a switch, and wherein the control circuitry is configured to open the switch and drain the at least one battery into the conductive plane.

14. The system of claim 1, wherein temporarily deactivating provision of negative pressure to the wound comprises preventing the negative pressure source from being activated to provide negative pressure to the wound for a first time period, and wherein the control circuitry is further configured to activate the negative pressure source to provide negative pressure to the wound responsive to expiration of the first time period.

15. The system of claim 14, wherein the first time period corresponds to a time period for clearing an error caused by one or more of the ingress of liquid or condensation onto the circuit board.

16. The system of claim 1, wherein permanently deactivating provision of negative pressure to the wound comprises preventing the negative pressure source from being activated to provide negative pressure to the wound.

17. The system of claim 1, wherein the control circuitry is further configured to detect the degradation of the electrical signal based on data obtained from one or more of:
a humidity sensor; or
an electronic fuse (eFuse).

18. The system of claim 1, wherein the control circuitry is configured to temporarily or permanently deactivate provision of negative pressure by one or more of:
blowing a fuse;
opening the fuse;
opening a switch; or
opening a relay.

19. The system of claim 1, wherein the control circuitry is further configured to provide an alarm responsive to the detection of the degradation of the electrical signal.

20. The system of claim 1, wherein the control circuitry comprises a programmable controller configured to execute instructions that detect the degradation of the electrical signal and, responsive to detection of the degradation of the electrical signal, temporarily or permanently deactivate provision of negative pressure to the wound.

21. A negative pressure wound therapy system comprising:
a negative pressure source configured to provide negative pressure to a wound covered by a wound dressing;
a printed circuit board;
electronic circuitry supported by the printed circuit board and configured to control operation of the negative pressure source; and
at least one fuse configured to provide overcurrent protection, the at least one fuse positioned on the printed circuit board and not surrounded by conductive material, thereby preventing formation of a heat sink for the at least one fuse.

22-43. (canceled)
